(19) United States
(12) Patent Application Publication
(10) Pub. No.: US 2004/0248739 A1
(43) Pub. Date: Dec. 9, 2004

(54) Pyridylpropynyloxyphenyl derivatives for use as herbicides

(76) Inventors: Jurgen Schaetzer, Basel (CH); Martin Eberle, Bottmingen (CH); Jean Wenger, Basel (CH); Sabine Berteina-Raboin, Orleans Cedex (FR); Kurt Nebel, Basel (CH); Andre Stoller, Basel (CH); Roger Graham Hall, Basel (CH); Steven Scott Bondy, Danville, CA (US); Daniel Dennis Comer, Spring Valley, CA (US); Julie Elizabeth Penzotti, Seattle, WA (US); Peter Diederik Jan Grootenhuis, San Diego, CA (US)

Correspondence Address: SYNGENTA CROP PROTECTION , INC., PATENT AND TRADEMARK DEPARTMENT, 410 SWING ROAD, GREENSBORO, NC, 27409, US

(21) Appl. No.: 10/486,271

(22) PCT Filed: Aug. 8, 2002
(86) PCT No.: PCT/EP02/08878

(30) Foreign Application Priority Data
Aug. 9, 2001 (CH) 1471/01

(51) Int. Cl. A01N 43/40; C07D 213/44

(52) U.S. Cl. 504/244; 546/290; 546/286; 546/315; 546/312; 546/339

(57) ABSTRACT

Compounds of formula (I), wherein the substituents R1, R2, R3, R4 and Z and the suffixes n and m are as defined in claim 1, and the agrochemically acceptable salts and all stereoisomers and tautomers of those compounds are suitable for use as herbicides.

The present invention relates to novel herbicidally active pyridyl-alkynes and pyridyl N-oxide-alkynes, to processes for their preparation, to compositions comprising those compounds, and to their use in controlling weeds, especially in crops of useful plants, or in inhibiting plant growth.

Phenylalkynes having herbicidal action are described, for example, in JP-A-11 147 866, WO 01/55066 and PCT Application No. EP01/11353.

Novel pyridyl-alkynes and pyridyl N-oxide-alkynes having herbicidal and growth-inhibiting properties have now been found.

The present invention accordingly relates to compounds of formula I

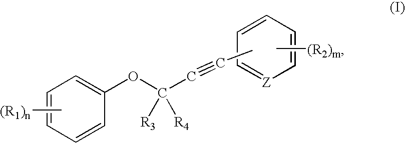

wherein

Z is ═N— or

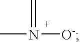

n is 0, 1, 2, 3, 4 or 5;

each R₁ independently of any others is halogen, —CN, —SCN, —SF₅, —NO₂, —NR₅R₆, —CO₂R₇, —CONR₈R₉, —C(R₁₀)═NOR₁₁, —COR₁₂, —OR₁₃, —SR₁₄, —SOR₁₅, —SO₂R₁₆, —OSO₂R₁₇, C₁-C₈alkyl, C₂-C₈alkenyl, C₂-C₈alkynyl or C₃-C₆cycloalkyl; or is C₁-C₈alkyl, C₂-C₈alkenyl or C₂-C₈alkynyl substituted by one or more halogen, —CN, —NO₂, —NR₁₈R₁₉, —CO₂R₂₀, —CONR₂₁R₂₂, —COR₂₃, —C(R₂₄)═NOR₂₅, —C(S)NR₂₆R₂₇, —C(C₁-C₄alkylthio)═NR₂₈, —OR₂₉, —SR₃₀, —SOR₃₁, —SO₂R₃₂ or C₃-C₆cycloalkyl substituents; or

each R₁ independently of any others is C₃-C₆cycloalkyl substituted by one or more halogen, —CN, —NO₂, —NR₁₈R₁₉, —CO₂R₂₀, —CONR₂₁R₂₂, —COR₂₃, —C(R₂₄)═NOR₂₅, —C(S)NR₂₆R₂₇, —C(C₁-C₄alkylthio)═NR₂₈, —SR₃₀, —SOR₃₁, —SO₂R₃₂ or C₃-C₆cycloalkyl substituents; or

each R₁ independently of any others is phenyl, which may in turn be substituted by one or more halogen, C₁-C₄alkyl, C₁-C₄haloalkyl, C₁-C₄alkoxy, —CN, —NO₂, C₁-C₄alkylthio, C₁-C₄alkyl-sulfinyl or C₁-C₄alkylsulfonyl substituents; or

two adjacent R₁ together form a C₁-C₇alkylene bridge, which may be interrupted by 1 or 2 non-adjacent oxygen atoms and may be substituted by C₁-C₆alkyl or C₁-C₆alkoxy, the total number of ring atoms being at least 5 and at most 9; or

two adjacent R₁ together form a C₂-C₇alkenylene bridge, which may be interrupted by 1 or 2 non-adjacent oxygen atoms and may be substituted by C₁-C₆alkyl or C₁-C₆alkoxy, the total number of ring atoms being at least 5 and at most 9;

R₃ and R₄ are each independently of the other hydrogen, halogen, —CN, C₁-C₄alkyl or C₁-C₄-alkoxy; or

R₃ and R₄ together are C₂-C₅alkylene;

R₅ is hydrogen or C₁-C₈alkyl;

R₆ is hydrogen, C₁-C₈alkyl, C₃-C₈alkenyl, C₃-C₈alkynyl, phenyl or benzyl; wherein phenyl and benzyl may in turn be substituted by one or more halogen, C₁-C₄alkyl, C₁-C₄haloalkyl, C₁-C₄alkoxy, —CN, —NO₂, C₁-C₄alkylthio, C₁-C₄alkylsulfinyl or C₁-C₄alkylsulfonyl substituents; or

R₅ and R₆ together are a C₂-C₅alkylene chain, which may be interrupted by an oxygen or a sulfur atom;

R₇ is hydrogen, C₁-C₈alkyl, C₃-C₈alkenyl or C₃-C₈alkynyl, or is CO—C₈alkyl, C₃-C₈alkenyl or C₃-C₈alkynyl substituted by one or more halogen, C₁-C₄alkoxy or phenyl substituents, wherein phenyl may in turn be substituted by one or more halogen, C₁-C₄alkyl, C₁-C₄halo-alkyl, C₁-C₄alkoxy, —CN, —NO₂, C₁-C₄alkylthio, C₁-C₄alkylsulfinyl or C₁-C₄alkylsulfonyl substituents;

R₈ is hydrogen or C₁-C₈alkyl;

R₉ is hydrogen or C₁-C₈alkyl, or is C₁-C₈alkyl substituted by one or more —COOH, C₁-C₈alkoxycarbonyl or —CN substituents, or

R₉ is C₃-C₈alkenyl, C₃-C₈alkynyl, phenyl or benzyl, wherein phenyl and benzyl may in turn be substituted by one or more halogen, C₁-C₄alkyl, C₁-C₄haloalkyl, C₁-C₄alkoxy, —CN, —NO₂, C₁-C₄alkylthio, C₁-C₄alkylsulfinyl or C₁-C₄alkylsulfonyl substituents; or

R₈ and R₉ together are C₂-C₅alkylene;

R₁₀ is hydrogen, C₁-C₄alkyl, C₁-C₄haloalkyl or C₃-C₆cycloalkyl;

R₁₁ is hydrogen, C₁-C₈alkyl, C₃-C₈alkenyl, C₃-C₈alkynyl, C₁-C₄haloalkyl or C₃-C₆haloalkenyl;

R₁₂ is hydrogen, C₁-C₄alkyl, C₁-C₄haloalkyl or C₃-C₆cycloalkyl;

R₁₃ is hydrogen, C₁-C₈alkyl, C₃-C₈alkenyl or C₃-C₈alkynyl; or

R₁₃ is phenyl or phenyl-C₁-C₆alkyl, wherein both phenyl rings may in turn be substituted by one or more halogen, C₁-C₄alkyl, C₁-C₄haloalkyl, C₁-C₄alkoxy, —CN, —NO₂, C₁-C₈alkylthio, C₁-C₈alkylsulfinyl or C₁-C₈alkylsulfonyl substituents, or

R₁₃ is C₁-C₈alkyl substituted by one or more halogen, —CN, C₁-C₆alkylamino, di(C₁-C₆alkyl)-amino or C₁-C₄alkoxy substituents;

R₁₄ is hydrogen, C₁-C₈alkyl, C₃-C₈alkenyl or C₃-C₈alkynyl, or is C₁-C₈alkyl substituted by one or more halogen, —CN or C₁-C₄alkoxy substituents;

R₁₅, R₁₆ and R₁₇ are each independently of the others C₁-C₈alkyl, C₃-C₈alkenyl or C₃-C₈-alkynyl, or C₁-C₈alkyl substituted by one or more halogen, —CN or C₁-C₄alkoxy substituents;

R₁₈ is hydrogen or C₁-C₈alkyl;

R₁₉ is hydrogen, C₁-C₈alkyl, C₃-C₈alkenyl, C₃-C₈alkynyl, phenyl or benzyl, wherein phenyl and benzyl may in turn be substituted by one or more halogen, C₁-C₄alkyl, C₁-C₄haloalkyl, C₁-C₄-alkoxy, —CN, —NO₂, C₁-C₄alkylthio, C₁-C₄alkylsulfinyl or C₁-C₄alkylsulfonyl substituents; or

R₁₈ and R₁₉ together are a C₂-C₅alkylene chain, which may be interrupted by an oxygen or a sulfur atom;

R₂₀ is hydrogen, C₁-C₈alkyl, C₃-C₈alkenyl, C₃-C₈alkynyl, phenyl or benzyl, wherein phenyl and benzyl may in turn be substituted by one or more halogen, C₁-C₄alkyl, C₁-C₄haloalkyl, C₁-C₄-alkoxy, —CN, —NO₂, C₁-C₄alkylthio, C₁-C₄alkylsulfinyl or C₁-C₄alkylsulfonyl substituents;

R₂₁ is hydrogen or C₁-C₈alkyl;

R₂₂ is hydrogen or C₁-C₈alkyl, or is C₁-C₈alkyl substituted by one or more —COOH, C₁-C₈-alkoxycarbonyl or —CN substituents, or

R₂₂ is C₃-C₈alkenyl, C₃-C₈alkynyl, phenyl or benzyl, wherein phenyl and benzyl may in turn be substituted by one or more halogen, C₁-C₄alkyl, C₁-C₄haloalkyl, C₁-C₄alkoxy, —CN, —NO₂, C₁-C₄alkylthio, C₁-C₄alkylsulfinyl or C₁-C₄alkylsulfonyl substituents; or

R₂₁ and R₂₂ together are C₂-C₅alkylene;

R₂₃ is hydrogen, C₁-C₄alkyl, C₁-C₄haloalkyl or C₃-C₆cycloalkyl;

R₂₄ is hydrogen, C₁-C₄alkyl, C₁-C₄haloalkyl or C₃-C₆cycloalkyl;

R₂₅ is hydrogen, C₁-C₈alkyl, C₃-C₈alkenyl, C₃-C₈alkynyl, C₁-C₄haloalkyl or C₃-C₆haloalkenyl;

R₂₆ is hydrogen or C₁-C₈alkyl;

R₂₇ is hydrogen or C₃-C₈alkyl, or is C₃-C₈alkyl substituted by one or more —COOH, C₁-C₈-alkoxycarbonyl or —CN substituents, or

R₂₇ is C₃-C₈alkenyl, C₃-C₈alkynyl, phenyl or benzyl, wherein phenyl and benzyl may in turn be substituted by one or more halogen, C₁-C₄alkyl, C₁-C₄haloalkyl, C₁-C₄alkoxy, —CN, —NO₂, C₁-C₄alkylthio, C₁-C₄alkylsulfinyl or C₁-C₄alkylsulfonyl substituents; or

R₂₆ and R₂₇ together are C₂-C₅alkylene;

R28 is hydrogen or C₁-C₈alkyl;

R₂₉ and R₃₀ are each independently of the other hydrogen, C₁-C₈alkyl, C₃-C₈alkenyl or C₃-C₈alkynyl, or C₁-C₈alkyl substituted by one or more halogen, —CN or C₁-C₄alkoxy substituents;

R₃₁ and R₃₂ are each independently of the other C₁-C₈alkyl, C₃-C₈alkenyl or C₃-C₈alkynyl, or C₁-C₈alkyl substituted by one or more halogen, —CN or C₁-C₄alkoxy substituents;

m is 0, 1, 2, 3 or 4;

each R₂ independently of any others is halogen, —CN, —SCN, —OCN, —N₃, —SF₅, —NO₂, —NR₃₃R34, —CO₂R₃₅, —CONR₃₆R₃₇, —C(R₃₈)═NOR₃₉, —COR₄₀, —OR₄₁, —SR₄₂, —SOR43, —SO₂R44, —OSO₂R45, —N([CO]ₚR₄₆)COR₄₇, —N(OR₅₄)COR₅₅, —N(R₅₆)SO₂R₅₇, —N(SO₂R₅₈)SO₂R₅₉, —N═C(OR₆₀)R₆₁, —CR₆₂(OR₆₃)OR₆₄, —OC(O)NR₆₅R₆₆, —SC(O)NR₆₇R₆₈, —OC(S)NR₆₉R₇₀ or —N-phthalimide; or

R₂ is a 5- to 7-membered heterocyclic ring system which may be aromatic or partially or fully saturated and may contain from 1 to 4 hetero atoms selected from nitrogen, oxygen and sulfur, it being possible for that heterocyclic ring system in turn to be substituted by one or more halogen, C₁-C₄alkyl, C₁-C₄haloalkyl, hydroxy-C₁-C₄alkyl, C₁-C₄alkoxy, C₁-C₄alkoxy-C₁-C₄alkyl, —CN, —NO₂, C₁-C₆alkylthio, C₁-C₆alkylsulfinyl or C₁-C₆alkylsulfonyl substituents;

R33 is hydrogen or C₁-C₈alkyl; and

R34 is hydrogen, C₁-C₈alkyl, C₃-C₈alkenyl, C₃-C₈alkynyl, phenyl or benzyl, wherein phenyl and benzyl may in turn be substituted by one or more halogen, C₁-C₄alkyl, C₁-C₄haloalkyl, C₁-C₄-alkoxy, —CN, —NO₂, C₁-C₄alkylthio, C₁-C₄alkylsulfinyl or C₁-C₄alkylsulfonyl substituents; or

R33 and R34 together are a C₂-C₅alkylene chain, which may be interrupted by an oxygen or a sulfur atom;

R₃₅ is hydrogen, C₁-C₈alkyl, C₃-C₈alkenyl or C₃-C₈alkynyl, or is C₁-C₈alkyl, C₃-C₈alkenyl or C₃-C₈alkynyl substituted by one or more halogen, C₁-C₄alkoxy or phenyl substituents, wherein phenyl may in turn be substituted by one or more halogen, C₁-C₄alkyl, C₁-C₄halo-alkyl, C₁-C₄alkoxy, —CN, —NO₂, C₁-C₄alkylthio, C₁-C₄alkylsulfinyl or C₁-C₄alkylsulfonyl substituents;

R₃₆ is hydrogen or C₁-C₈alkyl;

R₃₇ is hydrogen or C₁-C₈alkyl, or is C₁-C₈alkyl substituted by one or more —COOH, C₁-C₈-alkoxycarbonyl or —CN substituents, or

R₃₇ is C₃-C₈alkenyl, C₃-C₈alkynyl, phenyl or benzyl, wherein phenyl and benzyl may in turn be substituted by one or more halogen, C₁-C₄alkyl, C₁-C₄haloalkyl, C₁-C₄alkoxy, —CN, —NO₂, C₁-C₄alkylthio, C₁-C₄alkylsulfinyl or C₁-C₄alkylsulfonyl substituents; or

R₃₆ and R₃₇ together are C₃-C₅alkylene;

R₃₈ is hydrogen, C₁-C₄alkyl, C₁-C₄haloalkyl or C₃-C₆cycloalkyl;

R39 is hydrogen, C₁-C₈alkyl, C₃-C₈alkenyl, C₃-C₈alkynyl, C₁-C₄haloalkyl or C₃-C₆haloalkenyl;

R₄₀ is hydrogen, C₁-C₄alkyl, C₁-C₄haloalkyl, C₁-C₈alkylthio, —C(O)—C(O)OC₁-C₄alkyl or C₃-C₆-cycloalkyl;

R₄₁ is hydrogen, C₁-C₈alkyl, C₃-C₈alkenyl, C₃-C₈alkynyl, C₁-C₆alkoxy-C₁-C₆alkyl, C₁-C₈alkyl-carbonyl, C₁-C₈alkoxycarbonyl, C₃-C₈alkenyloxycarbonyl, C₁-C₆alkoxy-C₁-C₆alkoxycarbonyl, C₁-C₆alkylthio-C₁-C₆alkyl, C₁-C₆alkylsulfinyl-C₁-C₆alkyl or C₁-C₆alkylsulfonyl-C₁-C₆alkyl; or

R₄₁ is phenyl or phenyl-C₁-C₆alkyl, wherein both phenyl rings may in turn be substituted by one or more halogen, C₁-C₄alkyl, C₁-C₄haloalkyl, C₁-C₄alkoxy, —CN, —NO₂, or —S(O)₂C₁-C₈alkyl substituents, or

R₄₁ is C₁-C₈alkyl substituted by one or more —COOH, C₁-C₈alkoxycarbonyl, C₁-C₆alkylamino, di(C₁-C₆alkyl)amino or —CN substituents;

R₄₂ is hydrogen, C₁-C₈alkyl, C₃-C₈alkenyl or C₃-C₈alkynyl, or is C₁-C₈alkyl substituted by one or more halogen, —CN or C₁-C₄alkoxy substituents;

R43 and R₄₄ are each independently of the other C₁-C₈alkyl, C₃-C₈alkenyl or C₃-C₈alkynyl, or C₁-C₈alkyl substituted by one or more halogen, —CN or C₁-C₄alkoxy substituents;

R₄₅ is C₁-C₈alkyl, C₁-C₈alkyl substituted by one or more halogen, —CN or C₁-C₄alkoxy substituents, C₃-C₈alkenyl or C₃-C₈alkynyl, or

R₄₅ is phenyl, it being possible for the phenyl ring to be substituted by one or more halogen, C₁-C₄alkyl, C₁-C₄haloalkyl, C₁-C₄alkoxy, —CN, —NO₂, C₁-C₈alkylthio, C₁-C₈alkylsulfinyl or C₁-C₈alkylsulfonyl substituents;

R₄₆ is hydrogen, C₁-C₈alkyl, C₃-C₈alkenyl, C₃-C₈alkynyl or C₁-C₄haloalkyl;

R₄₇ is hydrogen, C₁-C₈alkyl, C₁-C₄alkoxy, C₃-C₈alkenyl or C₃-C₈alkynyl, or is C₁-C₈alkyl substituted by one or more halogen, —CN, C₁-C₄alkoxy, C₁-C₈alkoxycarbonyl, —NH₂, C₁-C₄-alkylamino, di(C₁-C₄-alkyl)amino, —NR₄₈COR₄₉, —NR₅₀SO₂R₅₁ or —NR₅₂CO₂R53 substituents, or R₄₇ is phenyl or benzyl, each of which may in turn be substituted by one or more halogen, C₁-C₄alkyl, C₁-C₄haloalkyl, C₁-C₄alkoxy, —CN, —NO₂, C₁-C₄alkylthio, C₁-C₄alkylsulfinyl or C₁-C₄alkylsulfonyl substituents;

p is 0 or 1;

R₄₈, R₄₉, R₅₀, R₅₁, R₅₂ and R₅₃ are each independently of the others hydrogen, C₁-C₈alkyl, phenyl, benzyl or naphthyl, it being possible for the three last-mentioned aromatic radicals in turn to be substituted by one or more halogen, C₁-C₈alkyl, C₁-C₄haloalkyl, C₁-C₄alkoxy, C₁-C₄alkylamino, di(C₁-C₄alkyl)amino, —NH₂, —CN, —NO₂, C₁-C₄alkylthio, C₁-C₄alkylsulfinyl or C₁-C₄alkylsulfonyl substituents;

R54 and R₅₅ are each independently of the other hydrogen, C₁-C₈alkyl or phenyl, whereby the phenyl ring may in turn be substituted by one or more halogen, C₁-C₄alkyl, C₁-C₄haloalkyl, C₁-C₄alkoxy, —CN, —NO₂, C₁-C₈alkylthio, C₁-C₈alkylsulfinyl or C₁-C₈alkylsulfonyl substituents;

R₅₆ is hydrogen, C₁-C₈alkyl, C₁-C₄haloalkyl, C₁-C₄alkoxy, C₃-C₈alkenyl, C₃-C₈alkynyl or benzyl, it being possible for benzyl in turn to be substituted by one or more halogen, C₁-C₄alkyl, C₁-C₄haloalkyl, C₁-C₄alkoxy, —CN, —NO₂, C₁-C₈alkylthio, C₁-C₈alkylsulfinyl or C₁-C₈alkylsulfonyl substituents;

R₅₇ is C₁-C₈alkyl, C₁-C₄haloalkyl, phenyl, benzyl or naphthyl, it being possible for the three last-mentioned aromatic rings to be substituted by one or more halogen, C₁-C₄alkyl, C₁-C₄haloalkyl, C₁-C₄alkoxy, C₁-C₄alkylamino, di(C₁-C₄alkyl)amino, —NH₂, —CN, —NO₂, C₁-C₄alkylthio, C₁-C₄alkylsulfinyl or C₁-C₄alkylsulfonyl substituents;

R₅₈ and R₅₉ are each independently of the other C₁-C₈alkyl, C₃-C₈alkenyl, C₃-C₈alkynyl, phenyl, benzyl or naphthyl, it being possible for the three last-mentioned aromatic rings to be substituted by one or more halogen, C₁-C₄alkyl, C₁-C₄haloalkyl, C₁-C₄alkoxy, C₁-C₄alkyl-amino, di(C₁-C₄alkyl)amino, —NH₂, —CN, —NO₂, C₁-C₄alkylthio, C₁-C₄alkylsulfinyl or C₁-C₄alkyl-sulfonyl substituents;

R₆₀ and R₆₁ are each independently of the other hydrogen or C₁-C₆alkyl;

R₆₂, R₆₃ and R64 are each independently of the others hydrogen or C₁-C₈alkyl, or

R₆₃ and R64 together form a C₂-C₅alkylene bridge;

R₆₅, R66, R₆₇, R68, R₆₉ and R₇₀ are each independently of the others hydrogen or C₁-C₈alkyl, or

R₆₅ and R₆₆ together or R₆₇ and R₆₈ together or R₆₉ and R₇₀ together form a C₂-C₅alkylene bridge; or

each R₂ independently of any others is C₁-C₈alkyl, or is C₁-C₈alkyl mono- or poly-substituted by halogen, —CN, —N₃, —SCN, —NO₂, —NR₇₁R₇₂, —CO₂R₇₃, —CONR₇₄R₇₅, —COR₇₆, —C(R77)═NOR₇₈, —C(S)NR₇₉R80, —C(C₁-C₄alkylthio)═NR₈₁, —OR₈₂, —SR₈₃, —SOR₈₄, —SO₂R₈₅, —O(SO₂)R₈₆, —N(R₈₇)CO₂R₈₈, —N(R₈₉)COR₉₀, —S⁺(R₉₁)₂, —N⁺(R₉₂)₃, —Si(R₉₃)₃ or C₃-C₆cycloalkyl; or

each R₂ independently of any others is C₁-C₈alkyl substituted by a 5- to 7-membered heterocyclic ring system, which may be aromatic or partially or fully saturated and may contain from 1 to 4 hetero atoms selected from nitrogen, oxygen and sulfur, it being possible for that heterocyclic ring system in turn to be substituted by one or more halogen, C₁-C₄alkyl, C₁-C₄haloalkyl, hydroxy-C₁-C₄alkyl, C₁-C₄alkoxy, C₁-C₄alkoxy-C₁-C₄alkyl, —CN, —NO₂, C₁-C₆alkylthio, C₁-C₆alkylsulfinyl or C₁-C₆alkylsulfonyl substituents; or

each R₂ independently of any others is C₂-C₈alkenyl, or is C₂-C₈alkenyl mono- or poly-substituted by halogen, —CN, —NO₂, —CO₂R₉₄, —CONR₉₅R₉₆, —COR₉₇, —C(R₉₈)═NOR₉₉, —C(S)NR₁₀OR₁₀₁, —C(C₁-C₄alkylthio)═NR₁₀₂, —OR₁₀₃, —Si(R₁₀₄)₃ or C₃-C₆cycloalkyl; or each R₂ independently of any others is C₂-C₈alkynyl, or is C₂-C₈alkynyl mono- or poly-substituted by halogen, —CN, —CO₂R₁₀₅, —CONR₁₀6R₁₀₇, —COR₁₀₈, —C(R₁₀₉)═NOR₁₁₀, —C(S)NR₁₁₁R112, —C(C₁-C₄alkylthio)═NR₁₁₃, —OR₁₁₄, —Si(R₁₁₅)₃ or C₃-C₆cycloalkyl; or

each R₂ independently of any others is C₃-C₆cycloalkyl, or is C₃-C₆cycloalkyl mono- or poly-substituted by halogen, —CN, —CO₂R₁₁₆, —CONR₁₁₇R₁₁₈, —COR₁₁₉, —C(R₁₂₀)═NOR₁₂₁, —C(S)NR₁₂₂R₁₂₃ or —C(C₁-C₄alkylthio)═NR₁₂₄; or

two adjacent R₂ together form a C₁-C₇alkylene bridge, which may be interrupted by 1 or 2 non-adjacent oxygen atoms and may be substituted by C₁-C₆alkyl or C₁-C₆alkoxy, the total number of ring atoms being at least 5 and at most 9; or

two adjacent R₂ together form a C₂-C₇alkenylene bridge, which may be interrupted by 1 or 2 non-adjacent oxygen atoms and may be substituted by C₁-C₆alkyl or C₁-C₆alkoxy, the total number of ring atoms being at least 5 and at most 9;

R₇₁ is hydrogen or C₁-C₈alkyl;

R₇₂ is hydrogen, C₁-C₈alkyl, C₃-C₈alkenyl, C₃-C₈alkynyl, phenyl or benzyl, wherein phenyl and benzyl may in turn be substituted by one or more halogen, C₁-C₄alkyl, C₁-C₄haloalkyl, C₁-C₄alkoxy, —CN, —NO₂, C₁-C₄alkylthio, C₁-C₄alkylsulfinyl or C₁-C₄alkylsulfonyl substituents; or

R₇₁ and R₇₂ together are a C₂-C₅alkylene chain, which may be interrupted by an oxygen or a sulfur atom;

R₇₃ is hydrogen, C₁-C₈alkyl, C₃-C₈alkenyl or C₃-C₈alkynyl, or is C₁-C₈alkyl, C₃-C₈alkenyl or C₃-C₈alkynyl substituted by one or more halogen, C₁-C₄alkoxy or phenyl substituents, it being possible for phenyl in turn to be substituted by one or more halogen, C₁-C₄alkyl, C₁-C₄haloalkyl, C₁-C₄alkoxy, —CN, —NO₂, C₁-C₄alkylthio, C₁-C₄alkylsulfinyl or C₁-C₄alkyl-sulfonyl substituents;

R₇₄ is hydrogen or C₁-C₈alkyl;

R₇₅ is hydrogen, C₁-C₈alkyl or C₃-C₇cycloalkyl, or is C₁-C₈alkyl substituted by one or more —COOH, C₁-C₈alkoxycarbonyl, C₁-C₆alkoxy or —CN substituents; or

R₇₅ is C₃-C₈alkenyl, C₃-C₈alkynyl, phenyl or benzyl, wherein phenyl and benzyl may in turn be substituted by one or more halogen, C₁-C₄alkyl, C₁-C₄haloalkyl, C₁-C₄alkoxy, —CN, —NO₂, C₁-C₄alkylthio, C₁-C₄alkylsulfinyl or C₁-C₄alkylsulfonyl substituents; or

R₇₄ and R₇₅ together are a C₂-C₅alkylene chain, which may be interrupted by an oxygen or sulfur atom;

R₇₆ is hydrogen, C₁-C₄alkyl, C₁-C₄haloalkyl or C₃-C₆cycloalkyl;

R77 is hydrogen, C₁-C₄alkyl, C₁-C₄haloalkyl or C₃-C₆cycloalkyl;

R₇₈ is hydrogen, C₁-C₈alkyl, C₃-C₈alkenyl, C₃-C₈alkynyl, C₁-C₄haloalkyl or C₃-C₆haloalkenyl; and

R₇₉ is hydrogen or C₁-C₈alkyl;

R₈₀ is hydrogen or CO—C₈alkyl, or is C₁-C₈alkyl substituted by one or more —COOH, C₁-C₈-alkoxycarbonyl or —CN substituents; or

R₈₀ is C₃-C₈alkenyl, C₃-C₈alkynyl, phenyl or benzyl, wherein phenyl and benzyl may in turn be substituted by one or more halogen, C₁-C₄alkyl, C₁-C₄haloalkyl, C₁-C₄alkoxy, —CN, —NO₂, C₁-C₄alkylthio, C₁-C₄alkylsulfinyl or C₁-C₄alkylsulfonyl substituents; or

R₇₉ and R₈₀ together are C₂-C₅alkylene;

R₈₁ is hydrogen or C₁-C₈alkyl;

R₈₂ is —Si(C₁-C₆alkyl)₃, C₃-C₈alkenyl, C₃-C₈alkynyl or C₁-C₈alkyl, whereby C₁-C₈alkyl is mono- or poly-substituted by halogen, —CN, —NH₂, C₁-C₆alkylamino, di(C₁-C₆alkyl)amino or C₁-C₄alkoxy;

R₈₃ is hydrogen, C₁-C₈alkyl, C₃-C₈alkenyl, C₃-C₈alkynyl or C₁-C₈alkyl, whereby C₁-C₈alkyl is mono- or poly-substituted by halogen, —CN, —NH₂, C₁-C₆alkylamino, di(C₁-C₆alkyl)amino or C₁-C₄alkoxy;

R84, R₈₅ and R₈₆ are each independently of the others C₁-C₈alkyl, C₃-C₈alkenyl or C₃-C₈-alkynyl, or C₁-C₈alkyl which is substituted by one or more halogen, —CN or C₁-C₄alkoxy substituents;

R₈₇ and R89 are each independently of the other hydrogen, C₁-C₈alkyl or C₁-C₈alkoxy;

R₈₈ is C₁-C₈alkyl;

R₉₀ is hydrogen or C₁-C₈alkyl;

R₉₁ is C₁-C₄alkyl;

R₉₂ and R₉₃ are each independently of the other C₁-C₆alkyl;

R94 is hydrogen, C₁-C₈alkyl, C₃-C₈alkenyl or C₃-C₈alkynyl, each of which may be mono- or poly-substituted by one or more halogen, C₁-C₄alkoxy or phenyl substituents, wherein phenyl may in turn be substituted by one or more halogen, C₁-C₄alkyl, C₁-C₄haloalkyl, C₁-C₄alkoxy, —CN, —NO₂, C₁-C₄alkylthio, C₁-C₄alkylsulfinyl or C₁-C₄alkylsulfonyl substituents;

R₉₅ is hydrogen or C₁-C₈alkyl;

R₉₆ is hydrogen or C₁-C₈alkyl, or is C₁-C₈alkyl substituted by one or more —COOH, C₁-C₈-alkoxycarbonyl or —CN substituents; or

R₉₆ is C₃-C₈alkenyl, C₃-C₈alkynyl, phenyl or benzyl, wherein phenyl and benzyl may in turn be substituted by one or more halogen, C₁-C₄alkyl, C₁-C₄haloalkyl, C₁-C₄alkoxy, —CN, —NO₂, C₁-C₄alkylthio, C₁-C₄alkylsulfinyl or C₁-C₄alkylsulfonyl substituents; or

R₉₅ and R₉₆ together are C₂-C₅alkylene;

R₉₇ and R₉₈ are each independently of the other hydrogen, C₁-C₄alkyl, C₁-C₄haloalkyl or C₃-C₆cycloalkyl;

R₉₉ is hydrogen, CO—C₈alkyl, C₃-C₈alkenyl, C₃-C₈alkynyl, C₁-C₄haloalkyl or C₃-C₆haloalkenyl;

R₁₀₀ is hydrogen or C₁-C₈alkyl;

R₁₀₁ is hydrogen or C₁-C₈alkyl, or is C₁-C₈alkyl substituted by one or more —COOH, C₁-C₈-alkoxycarbonyl or —CN substituents; or

R₁₀₁ is C₃-C₈alkenyl, C₃-C₈alkynyl, phenyl or benzyl, wherein phenyl and benzyl may in turn be substituted by one or more halogen, C₁-C₄alkyl, C₁-C₄haloalkyl, C₁-C₄alkoxy, —CN, —NO₂, C₁-C₄alkylthio, C₁-C₄alkylsulfinyl or C₁-C₄alkylsulfonyl substituents; or

R₁₀₀ and R101 together are C₂-C₅alkylene;

R₁₀₂ is hydrogen or C₁-C₈alkyl;

R₁₀₃ is hydrogen, C₁-C₈alkyl, —Si(C₁-C₆alkyl)₃, C₃-C₈alkenyl or C₃-C₈alkynyl;

R₁₀₄ is C₁-C₆alkyl;

R₁₀₅ is hydrogen, C₁-C₈alkyl, C₃-C₈alkenyl or C₃-C₈alkynyl, each of which may be mono- or poly-substituted by one or more halogen, C₁-C₄alkoxy or phenyl substituents, wherein phenyl may in turn be substituted by one or more halogen, C₁-C₄alkyl, C₁-C₄haloalkyl, C₁-C₄alkoxy, —CN, —NO₂, C₁-C₄alkylthio, C₁-C₄alkylsulfinyl or C₁-C₄alkylsulfonyl substituents;

R₁₀₆ is hydrogen or C₁-C₈alkyl;

R₁₀₇ is hydrogen or C₁-C₈alkyl, or is C₁-C₈alkyl substituted by one or more —COOH, C₁-C₈-alkoxycarbonyl or —CN substituents; or

R₁₀₇ is C₃-C₈alkenyl, C₃-C₈alkynyl, phenyl or benzyl, wherein phenyl and benzyl may in turn be substituted by one or more halogen, C₁-C₄alkyl, C₁-C₄haloalkyl, C₁-C₄alkoxy, —CN, —NO₂, C₁-C₄alkylthio, C₁-C₄alkylsulfinyl or C₁-C₄alkylsulfonyl substituents; or

R₁₀₆ and R₁₀₇ together are C₂-C₅alkylene;

R₁₀₈ is hydrogen, C₁-C₄alkyl, C₁-C₄haloalkyl or C₃-C₆cycloalkyl;

R₁₀₉ is hydrogen, C₁-C₄alkyl, C₁-C₄haloalkyl or C₃-C₆cycloalkyl;

R110 is hydrogen, C₁-C₈alkyl, C₃-C₈alkenyl, C₃-C₈alkynyl, C₁-C₄haloalkyl or C₃-C₆haloalkenyl;

R₁₁₁ is hydrogen or C₁-C₈alkyl;

R₁₁₂ is hydrogen or C₁-C₈alkyl, or is C₁-C₈alkyl substituted by one or more —COOH, C₁-C₈-alkoxycarbonyl or —CN substituents; or

R₁₁₂ is C₃-C₈alkenyl, C₃-C₈alkynyl, phenyl or benzyl, wherein phenyl and benzyl may in turn be substituted by one or more halogen, C₁-C₄alkyl, C₁-C₄haloalkyl, C₁-C₄alkoxy, —CN, —NO₂, C₁-C₄alkylthio, C₁-C₄alkylsulfinyl or C₁-C₄alkylsulfonyl substituents; or

R₁₁₁ and R₁₁₂ together are C₂-C₅alkylene;

R₁₁₃ is hydrogen or C₁-C₈alkyl;

R₁₁₄ is hydrogen, C₁-C₈alkyl, —Si(C₁-C₆alkyl)₃, C₃-C₈alkenyl or C₃-C₈alkynyl;

R₁₁₅ is O₁—C₆alkyl;

R₁₁₆ is hydrogen, C₁-C₈alkyl, C₃-C₈alkenyl or C₃-C₈alkynyl, each of which may be mono- or poly-substituted by one or more halogen, C₁-C₄alkoxy or phenyl substituents, wherein phenyl may in turn be substituted by one or more halogen, C₁-C₄alkyl, C₁-C₄haloalkyl, C₁-C₄alkoxy, —CN, —NO₂, C₁-C₄alkylthio, C₁-C₄alkylsulfinyl or C₁-C₄alkylsulfonyl substituents;

R₁₁₇ is hydrogen or C₁-C₈alkyl;

R₁₁₈ is hydrogen or C₁-C₈alkyl, or is C₁-C₈alkyl substituted by one or more —COOH, C₁-C₈alkoxycarbonyl or —CN substituents; or

R₁₁₈ is C₃-C₈alkenyl, C₃-C₈alkynyl, phenyl or benzyl, wherein phenyl and benzyl may in turn be substituted by one or more halogen, C₁-C₄alkyl, C₁-C₄haloalkyl, C₁-C₄alkoxy, —CN, —NO₂, C₁-C₄alkylthio, C₁-C₄alkylsulfinyl or C₁-C₄alkylsulfonyl substituents; or

R₁₁₇ and R₁₁₈ together are C₂-C₅alkylene;

R₁₁₉ is hydrogen, C₁-C₄alkyl, C₁-C₄haloalkyl or C₃-C₆cycloalkyl;

R₁₂₀ is hydrogen, C₁-C₄alkyl, C₁-C₄haloalkyl or C₃-C₆cycloalkyl;

R₁₂₁ is hydrogen, C₁-C₈alkyl, C₃-C₈alkenyl, C₃-C₈alkynyl, C₁-C₄haloalkyl or C₃-C₆haloalkenyl;

R₁₂₂ is hydrogen or C₁-C₈alkyl;

R₁₂₃ is hydrogen or C₁-C₈alkyl, or is C₁-C₈alkyl substituted by one or more —COOH, C₁-C₈-alkoxycarbonyl or —CN substituents; or

R₁₂₃ is C₃-C₈alkenyl, C₃-C₈alkynyl, phenyl or benzyl, wherein phenyl and benzyl may in turn be substituted by one or more halogen, C₁-C₄alkyl, C₁-C₄haloalkyl, C₁-C₄alkoxy, —CN, —NO₂, C₁-C₄alkylthio, C₁-C₄alkylsulfinyl or C₁-C₄alkylsulfonyl substituents; or

R₁₂₂ and R₁₂₃ together are C₂-C₅alkylene; and

R₁₂₄ is hydrogen or C₁-C₈alkyl, and to the agrochemically acceptable salts and all stereoisomers and tautomers of the compounds of formula I.

When n is 0, all the free valencies on the phenyl ring of the compounds of formula I are substituted by hydrogen. When m is 0, all the free valencies on the pyridyl ring of the compounds of formula I are substituted by hydrogen.

Examples of substituents that are formed when R₅ and R₆ together or R₁₈ and R₁₉ together or R₃₆ and R₃₇ together or R₇₄ and R₇₅ together are a C₂-C₅alkylene chain, which may be interrupted by an oxygen or a sulfur atom, are piperidine, morpholine, thiomorpholine and pyrrolidine.

Examples of heterocyclic ring systems, which may be aromatic or partially or fully saturated, in the definition of R₂ are:

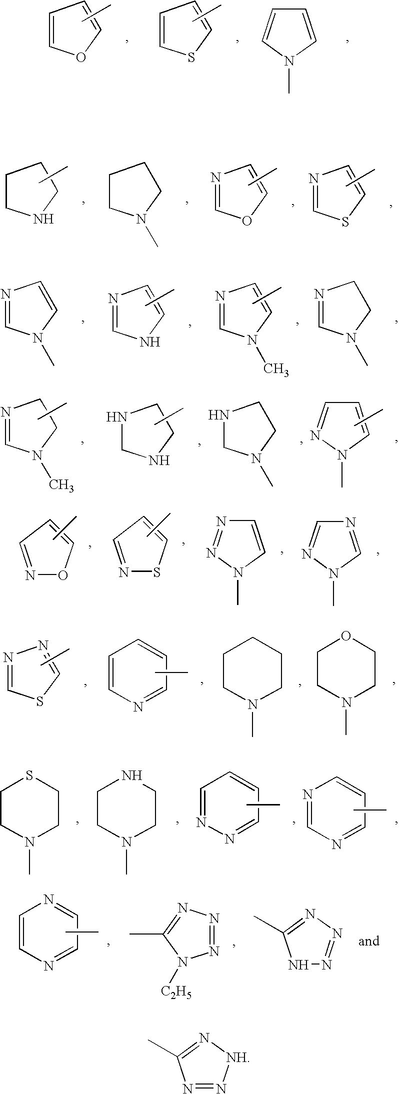

The alkyl groups appearing in the definitions of substituents may be straight-chain or branched and are, for example, methyl, ethyl, n-propyl, isopropyl, n-butyl, sec-butyl, isobutyl, tert-butyl, and also the isomers of pentyl, hexyl, heptyl, octyl, nonyl and decyl.

Halogen is fluorine, chlorine, bromine and iodine, preferably fluorine and chlorine.

Haloalkyl is, for example, fluoromethyl, difluoromethyl, trifluoromethyl, chloromethyl, dichloro-methyl, trichloromethyl, 2,2,2-trifluoroethyl, 2-fluoroethyl, 2-chloroethyl, pentafluoroethyl, 1,1-difluoro-2,2,2-trichloroethyl, 2,2,3,3-tetrafluoroethyl and 2,2,2-trichloroethyl; preferably tri-chloromethyl, difluorochloromethyl, difluoromethyl, trifluoromethyl and dichlorofluoromethyl.

Alkoxy groups have preferably a chain length of from 1 to 6, especially from 1 to 4, carbon atoms. Alkoxy is, for example, methoxy, ethoxy, propoxy, isopropoxy, n-butoxy, isobutoxy, sec-butoxy and tert-butoxy, and also the pentyloxy and hexyloxy isomers; preferably methoxy and ethoxy.

Alkoxy, alkenyl, alkynyl, alkoxyalkyl, alkylthio, alkylsulfonyl, alkylsulfinyl, alkylaminoalkoxy, alkoxycarbonyl, alkylcarbonyloxy, alkenylthio, alkenylsulfonyl, alkenylsulfinyl, alkynylsulfonyl, alkynylthio and alkynylsulfinyl groups are derived from the mentioned alkyl radicals. The alkenyl and alkynyl groups can be mono- or poly-unsaturated. Alkenyl is to be understood as being, for example, vinyl, allyl, methallyl, 1-methylvinyl or but-2-en-1-yl. Alkynyl is, for example, ethynyl, propargyl, but-2-yn-1-yl, 2-methylbutyn-2-yl or but-3-yn-2-yl.

Alkylthio groups have preferably a chain length of from 1 to 4 carbon atoms. Alkylthio is, for example, methylthio, ethylthio, propylthio, isopropylthio, n-butylthio, isobutylthio, sec-butylthio or tert-butylthio, preferably methylthio and ethylthio. Alkylsulfinyl is, for example, methyl-sulfinyl, ethylsulfinyl, propylsulfinyl, isopropylsulfinyl, n-butylsulfinyl, isobutylsulfinyl, sec-butylsulfinyl or tert-butylsulfinyl; preferably methylsulfinyl or ethylsulfinyl. Alkylsulfonyl is, for example, methylsulfonyl, ethylsulfonyl, propylsulfonyl, isopropylsulfonyl, n-butylsulfonyl, isobutylsulfonyl, sec-butylsulfonyl or tert-butylsulfonyl; preferably methyl-sulfonyl or ethylsulfonyl.

Alkoxyalkyl groups have preferably from 1 to 6 carbon atoms. Alkoxyalkyl is, for example, methoxymethyl, methoxyethyl, ethoxymethyl, ethoxyethyl, n-propoxymethyl, n-propoxyethyl, isopropoxymethyl or isopropoxyethyl.

Substituents wherein two adjacent R₁ together form a C₁-C₇alkylene bridge which may be interrupted by 1 or 2 non-adjacent oxygen atoms and may be substituted by C₁-C₆alkyl or C₁-C₆alkoxy, the total number of ring atoms being at least 5 and at most 9, or two adjacent R₁ together form a C₂-C₇alkenylene bridge which may be interrupted by 1 or 2 non-adjacent oxygen atoms and may be substituted by C₁-C₆alkyl or C₁-C₆alkoxy, the total number of ring atoms being at least 5 and at most 9, have, for example, the following structures:

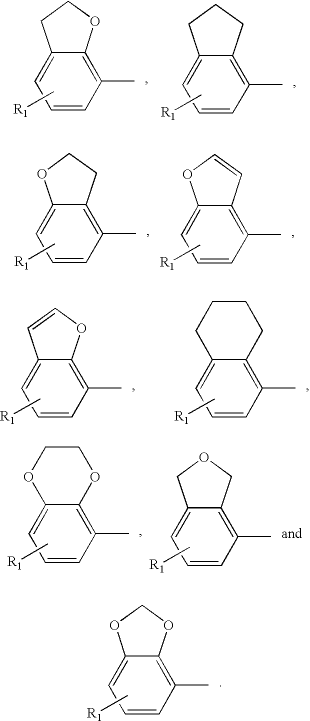

Substituents wherein two adjacent R₂ together form a C₁-C₇alkylene bridge which may be interrupted by 1 or 2 non-adjacent oxygen atoms and may be substituted by C₁-C₆alkyl or C₁-C₆alkoxy, the total number of ring atoms being at least 5 and at most 9, or two adjacent R₂ together form a C₂-C₇alkenylene bridge which may be interrupted by 1 or 2 non-adjacent oxygen atoms and may be substituted by C₁-C₆alkyl or C₁-C₆alkoxy, the total number of ring atoms being at least 5 and at most 9, have, for example, the following structures:

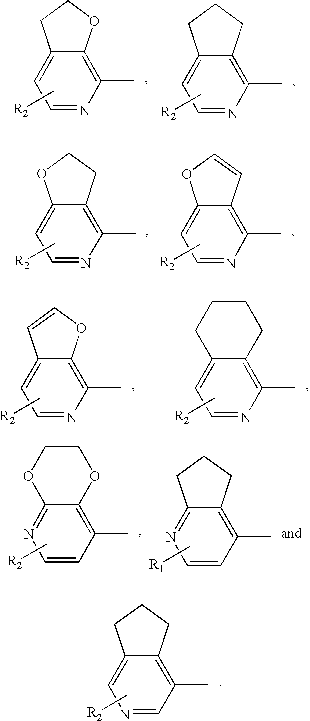

The invention relates also to the salts which the compounds of formula I are able to form especially with amines, alkali metal and alkaline earth metal bases or quaternary ammonium bases. Suitable salt-formers are described, for example, in WO 98/41089.

Among the alkali metal and alkaline earth metal hydroxides as salt formers, special mention should be made of the hydroxides of lithium, sodium, potassium, magnesium and calcium, but especially the hydroxides of sodium and potassium.

Examples of amines suitable for ammonium salt formation include ammonia as well as primary, secondary and tertiary C₁-C₁₈alkylamines, C₁-C₄hydroxyalkylamines and C₂-C₄-alkoxyalkylamines, for example methylamine, ethylamine, n-propylamine, isopropylamine, the four butylamine isomers, n-amylamine, isoamylamine, hexylamine, heptylamine, octyl-amine, nonylamine, decylamine, pentadecylamine, hexadecylamine, heptadecylamine, octa-decylamine, methylethylamine, methylisopropylamine, methylhexylamine, methylnonylamine, methylpentadecylamine, methyloctadecylamine, ethylbutylamine, ethylheptylamine, ethyl-octylamine, hexylheptylamine, hexyloctylamine, dimethylamine, diethylamine, di-n-propyl-amine, diisopropylamine, di-n-butylamine, di-n-amylamine, diisoamylamine, dihexylamine, diheptylamine, dioctylamine, ethanolamine, n-propanolamine, isopropanolamine, N,N-diethanolamine, N-ethylpropanolamine, N-butylethanolamine, allylamine, n-butenyl-2-amine, n-pentenyl-2-amine, 2,3-dimethylbutenyl-2-amine, dibutenyl-2-amine, n-hexenyl-2-amine, propylenediamine, trimethylamine, triethylamine, tri-n-propylamine, triisopropylamine, tri-n-butylamine, triisobutylamine, tri-sec-butylamine, tri-n-amylamine, methoxyethylamine and ethoxyethylamine; heterocyclic amines, for example pyridine, quinoline, isoquinoline, morpholine, piperidine, pyrrolidine, indoline, quinuclidine and azepine; primary arylamines, for example anilines, methoxyanilines, ethoxyanilines, o-, m- and p-toluidines, phenylenediamines, benzidines, naphthylamines and o-, m- and p-chloroanilines; but especially triethylamine, isopropylamine and diisopropylamine.

Preferred quaternary ammonium bases suitable for salt formation correspond e.g. to the formula [N(RₐR_{b}R_{c}R_{d})]OH wherein Rₐ, R_{b}, R_{c} and R_{d} are each independently of the other C₁-C₄alkyl. Other suitable tetraalkylammonium bases with other anions can be obtained, for example, by anion exchange reactions.

Preferred compounds of formula I are those wherein Z is ═N—; and each R₂ independently of any others is C₂-C₈alkenyl, or is C₂-C₈alkenyl mono- or poly-substituted by —CN, —NO₂, —CO₂R₉₄, —CONR₉₅R₉₆, —COR₉₇, —C(R₉₈)═NOR₉₉, —C(S)NR₁₀₀R₁₀₁, —C(C₁-C₄alkylthio)═NR₁₀₂, —OR₁₀₃, —Si(R₁₀₄)₃ or C₃-C₆cycloalkyl.

Further preferred compounds of formula I are those wherein each R₂ independently of any others is halogen, —CN, —SCN, —OCN, —N₃, —CONR₃₆R₃₇, —C(R₃₈)═NOR₃₉, —COR₄₀, —OR₄₁, —SO₂R4, —N([CO]ₚR46)COR₄₇, —N(R₅₆)SO₂R₅₇, —N(SO₂R₅₈)SO₂R₅₉, —N═C(OR₆₀)R₆₁ or C₁-C₈alkyl, or is C₁-C₈alkyl mono- or poly-substituted by halogen, —CN, —N₃, —SCN, —CONR₇₄R₇₅, —COR₇₆, —C(R77)═NOR₇₈, —C(S)NR₇₉R₈₀, —OR₈₂, —SOR84, —SO₂R₈₅ or —N(R₈₉)COR₉₀.

Preference is likewise given to compounds of formula I wherein each R₁ independently of any others is halogen, —CN, C₁-C₃alkyl, C₁-C₃haloalkyl, C₁-C₃cyanoalkyl, —OR₁₃ or —C(R₂₄)═NOR₂₅; R₁₃ is C₁-C₃alkyl or di(C₁-C₄-alkyl)amino-C₁-C₄alkyl; R₂₄ is hydrogen or methyl; and R₂₅ is hydrogen or C₁-C₃alkyl.

Also of importance are compounds of formula I wherein R₃ and R₄ are each independently of the other hydrogen or methyl.

The compounds of formula I can be prepared by methods known per se described, for example, in Tetrahedron 1997 (53), 12621-12628; Helv. Chim. Acta 2000 (83), 650-657; J. Chem. Res., Synop. 1996 (10), 462-463; Org. Prep. Proc. Int. 1995 (27), 129-160; Tetra-hedron Organic Chemistry 2000 (20), 209-213; and K. Sonogashira in “Comprehensive Organic Synthesis”, Editors 1. Fleming et al., Pergamon, Oxford 1991, Vol. 3, page 521 ff., for example by reacting a compound of formula II

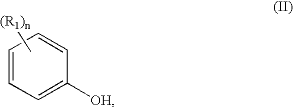

wherein R₁ and n are as defined for formula I, in the presence of a base, with a compound of formula III

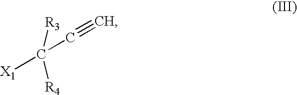

wherein R₃ and R₄ are as defined for formula I and XI is O-tosyl, O-mesyl, chlorine, bromine or iodine, to form a compound of formula IV

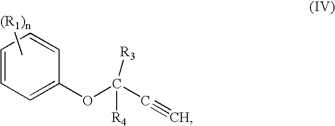

wherein R₁, R₃, R₄ and n are as defined, and then coupling that compound with a compound of formula V or Va

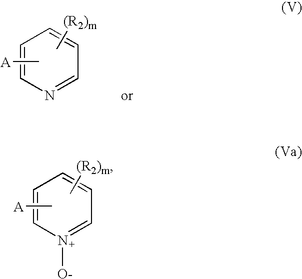

wherein R₂ and m are as defined for formula I and A is a leaving group, e.g. halogen or trifluoromethanesulfonate, in the presence of a palladium catalyst, and, if desired, oxidising the resulting pyridine derivative of formula I wherein Z is ═N— to form the corresponding pyridine N-oxide of formula I wherein Z is

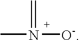

The preparation of the compounds of formula I can be carried out e.g. according to the individual Schemes 1, 2, 3, 4 and 5. For the individual synthesis schemes it is generally true that various substituents R₂ in a compound of formula V or Va are either already present at the outset or can be introduced in succession, for example by nucleophilic or electrophilic aromatic substitution.

Similarly, the compound of formula V may at the outset already be in the form of the pyridine N-oxide derivative of formula Va

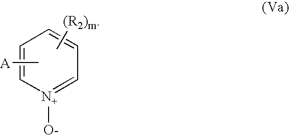

If desired, however, the N-oxide function can be introduced into the pyridyl ring of the compound of formula I wherein Z is ═N— only at the end of the synthesis sequence, via oxidation by conventional methods, e.g. with hydrogen peroxide or organic peracids.

According to Reaction Scheme 1, the compounds of formula I can be obtained, for example, from substituted phenyl propargyl ethers of formula IV.

The propargyl ethers of formula IV can be obtained beforehand by etherification of phenols of formula II, which are reacted in the presence of a base with acetylene derivatives of formula III. Such etherification reactions are standard procedures and can be carried out e.g. analogously to Tetrahedron 1997 (53), 12621-12628; Helv. Chim. Acta 2000 (83), 650-657; and J. Chem. Res., Synop. 1996 (10), 462-463.

In the next step, the propargyl ethers of formula IV are coupled with substituted pyridine or pyridine N-oxide derivatives of formula V or Va, respectively, under typical Sonogashira conditions (K. Sonogashira in “Comprehensive Organic Synthesis”, Editors I. Fleming et al., Pergamon, Oxford 1991, Vol. 3, page 521 ff.; J. Org. Chem. 1998 (63), 8551-8553). Catalyst mixtures that come into consideration are, for example, tetrakistriphenylphosphine-palladium or bistriphenylphosphine-palladium dichloride together with copper iodide, and bases that come into consideration (for the reductive elimination) are especially amines, for example triethylamine, diethylamine and diisopropylethylamine.

The pyridines or pyridine N-oxides of formula V or Va, respectively, preferably carry a leaving group A, wherein A is e.g. halogen or trifluoromethanesulfonate (Tetrahedron Organic Chemistry 2000 (20), 209-213; J. Org. Chem. 1997 (62), 1491-1500). As solvents for the Sonogashira reaction there are customarily used ethers, for example tetrahydrofuran, chlorinated hydrocarbons, for example chloroform, or dipolar aprotic solvents, for example dimethylformamide or dimethyl sulfoxide, or amines, for example triethylamine or piperidine.

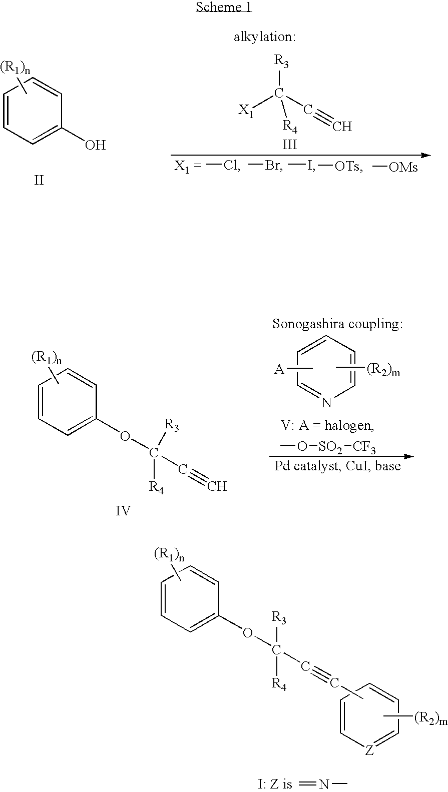

The Pd-catalysed cross-coupling of suitably substituted pyridine or pyridine N-oxide derivatives of formula V or Va, respectively, with propargyl alcohols or terminal acetylenes of formula VI

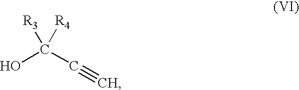

wherein R₃ and R₄ are as defined for formula I, is known generally as the Sonogashira reaction and is shown diagrammatically in Reaction Scheme 2 for the pyridine derivatives of formula V. That reaction is documented in detail in Tetrahedron Organic Chemistry 2000 (20), 209-213 and can be used for the preparation of the pyridyl and pyridyl N-oxide propargyl alcohols of formula VII

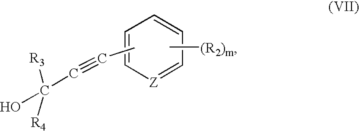

wherein R₂, R₃, R₄, Z and m are as defined for formula I.

The activation of the alcohol of formula VII (Z is ═N—) is carried out e.g. by sulfonylation or halogenation according to Scheme 2. The sulfonylation of the alcohol of formula VII is a standard reaction and can be carried out e.g. with a sulfonic acid chloride, for example mesyl chloride (MsCl) or para-toluenesulfonic acid chloride (p-TsCl), in the presence of a tertiary amine, for example triethylamine, or an aromatic amine, for example pyridine, in a solvent, e.g. a chlorinated hydrocarbon, for example carbon tetrachloride or methylene chloride, or an amine, for example pyridine. Such reactions are generally known and are described e.g. in J. Org. Chem. 1997 (62), 8987; J. Het. Chem. 1995 (32), 875-882; and also in Tetra-hedron Left. 1997 (38), 8671-8674.

The halogenation of the alcohol of formula VII (Z is ═N—) can be carried out analogously to standard procedures. For example, the bromination is carried out with carbon tetrabromide in the presence of triphenylphosphine (Synthesis 1998, 1015-1018) in methylene chloride. The chlorination is carried out with mineral acids, for example with concentrated hydrochloric acid (J. Org. Chem. 1955 (20), 95) or with para-toluenesulfonic acid chloride in the presence of an amine, for example triethylamine in a solvent, e.g. methylene chloride (Tetrahedron Left. 1984 (25), 2295).

The preparation of the pyridyl-propynyloxy-benzenes of formula I (Z is ═N—) can be carried out analogously to Synthesis 1995, 707-712; and Tetrahedron Left. 1994 (35), 6405-6408 by means of copper-iodide-catalysed etherification of the phenol of formula II in the presence of the tosylate or mesylate or halide of formula VIII (according to Scheme 2). Suitable solvents are dimethylformamide and acetonitrile, and suitable bases are especially potassium carbonate and 1,8-diazabicyclo[5.4.0]undec-7-ene (DBU).

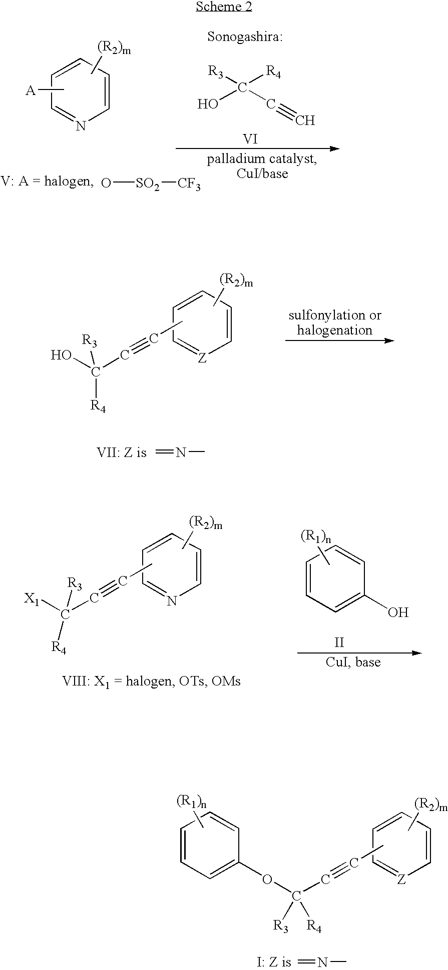

Compounds of formula I can also be obtained by further methods (according to Scheme 3).

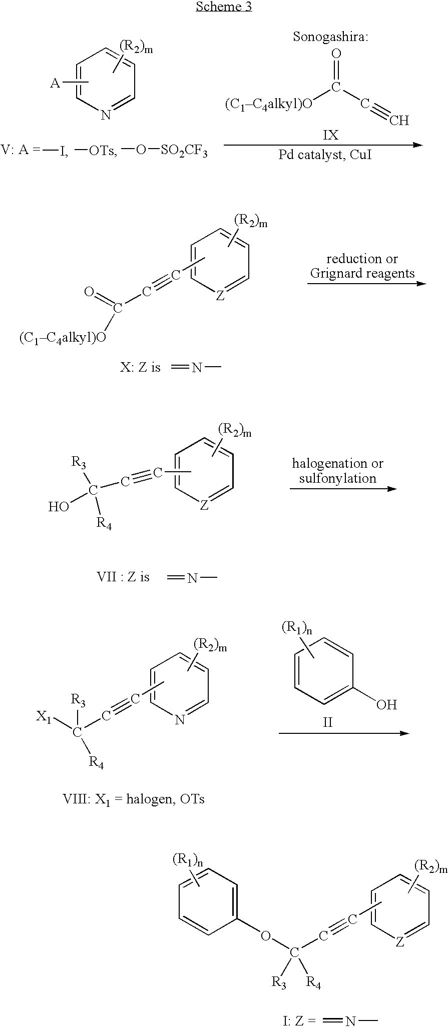

Accordingly, acetylene esters of formula X

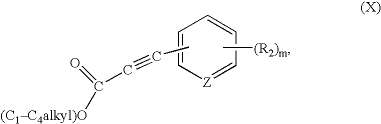

wherein R₂, Z and m are as defined for formula I, can be obtained, by means of Sonogashira coupling, from the compounds of formula IX

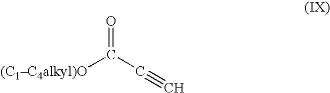

and activated pyridine derivatives of formula V or Va

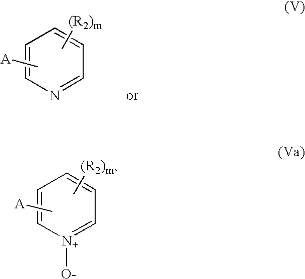

wherein R₂ and m are as defined and A is a leaving group as described above, analogously to Synthetic Communic. 1998 (28), 327-335. The esters of formula X can then be reduced or reacted with organometallic compounds, for example Grignard reagents, to form the alcohols of formula VII

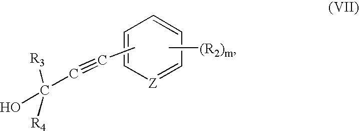

wherein R₂, Z and m are as defined for formula I and R3 and R₄ are each independently of the other hydrogen, C₁-C₄alkyl or C₁-C₄alkoxy.

The reduction of the acetylene esters of formula X (Z is ═N—) to the alcohols of formula VII (Z is ═N—) can be carried out especially with hydrides by standard methods, for example with lithium aluminium hydride or sodium borohydride in a solvent, e.g. an ether, for example diethyl ether, dioxane or tetrahydrofuran, or an alcohol, for example methanol or ethanol. Such reductions are described e.g. in C. Ferri, “Reaktionen der organischen Synthese” 1978, pages 98-102.

Reactions of carboxylic acid esters with Grignard reagents are standard in organic synthesis chemistry and are described in detail in “Organikum” 1976, pages 617-625. The subsequent etherification of the phenol derivatives of formula II in the presence of a compound of formula VIII to form the compounds of formula I has already been described in detail in Scheme 2.

Further methods of preparing the desired compounds of formula I are shown in Scheme 4 (variant of Scheme 3).

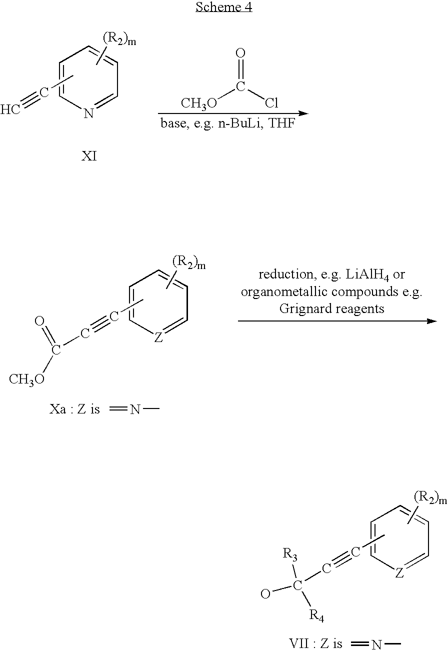

Accordingly, a pyridylacetylene of formula XI

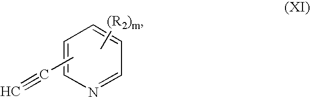

wherein R₂ and m are as defined for formula I, is reacted with n-butyllithium (n-BuLi) and then with a chloroformic acid methyl ester to form an ester of formula Xa

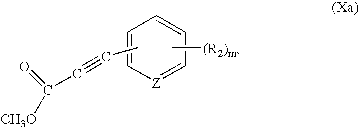

wherein Z is ═N—.

That ester can be converted into the desired compound of formula I entirely analogously to the method already described in Scheme 3, via an alcohol of formula VII (Z is ═N—) (analogously to J. Org. Chem. 1988 (53), 4166-4171).

The compounds of formula I can also be prepared by first reacting the propargyl alcohols of formula VI

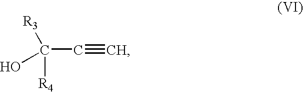

wherein R₃ and R₄ are as defined for formula I, with activated phenyl halides of formula XII

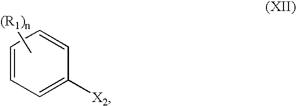

wherein X₂ is halogen, n is 1, 2, 3, 4 or 5 and R₁ is a substituent having an electron-withdrawing effect (-M and/or —I effect), e.g. —NO₂, —CN, CF₃ or COR₁₂, to form compounds of formula IV

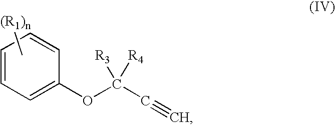

wherein R₁, R₃, R₄ and n are as defined, and then in the next synthesis step carrying out a Sonogashira reaction with activated pyridine or pyridine N-oxide derivatives of formula V or Va

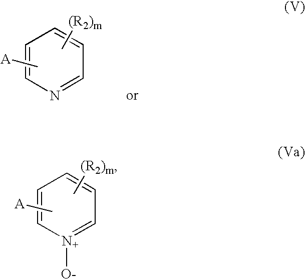

wherein R₂ and m are as defined for formula I and A is a leaving group, e.g. halogen or trifluoromethanesulfonate (Reaction Scheme 5).

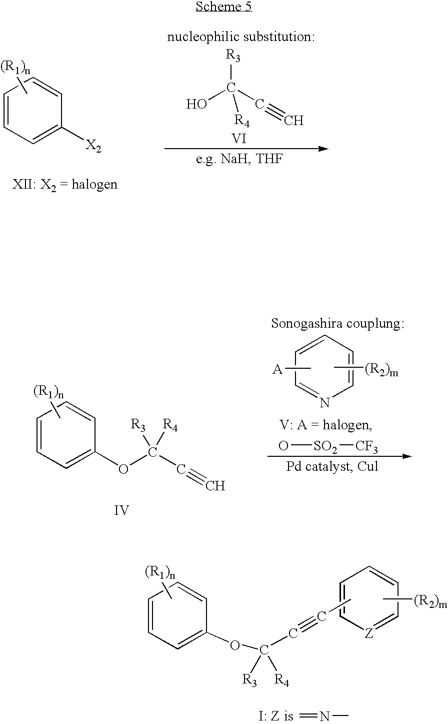

The following comments apply to the individual reaction steps in Schemes 1 to 5: The reactions to form compounds of formula I are advantageously performed in aprotic, inert organic solvents. Such solvents are hydrocarbons, such as benzene, toluene, xylene or cyclohexane, chlorinated hydrocarbons, such as dichloromethane, trichloromethane, tetra-chloromethane and chlorobenzene, ethers, such as diethyl ether, ethylene glycol dimethyl ether, diethylene glycol dimethyl ether, tetrahydrofuran and dioxane, nitrites, such as acetonitrile and propionitrile, amides, such as N,N-dimethylformamide, diethylformamide and N-methylpyrrolidinone. The reaction temperatures are preferably from −20° C. to +120° C. The reactions generally proceed slightly exothermically and can generally be carried out at room temperature. In order to shorten the reaction time or alternatively to initiate the reaction, the reaction mixture may, if appropriate, be heated to its boiling point for a short time. The reaction times may likewise be shortened by the addition of a few drops of base as reaction catalyst. Suitable bases are especially tertiary amines, such as trimethylamine, triethylamine, quinuclidine, 1,4diazabicyclo[2.2.2]octane, 1,5-diazabicyclo[4.3.0]non-5-ene and 1,5-diaza-bicyclo[5.4.0]undec-7-ene, but it is also possible to use inorganic bases, such as hydrides, e.g. sodium or calcium hydride, hydroxides, such as sodium or potassium hydroxide, carbonates, such as sodium or potassium carbonate, or hydrogen carbonates, such as potassium or sodium hydrogen carbonate.

The compounds of formula I can be isolated in customary manner by concentration and/or evaporation of the solvent and can be purified by recrystallisation or trituration of the solid residue in solvents in which they are not readily soluble, such as ethers, aromatic hydrocarbons or chlorinated hydrocarbons.

The starting compounds of formulae II, III, V, VI, IX, XI and XII used in Schemes 1 to 5 are known, in some cases are commercially available or can be prepared analogously to described standard methods. For example, the compounds of formula V are described in Tetrahedron Organic Chemistry 20, 209 (2000).

For the use according to the invention of the compounds of formula I, or of compositions comprising them, there come into consideration all methods of application customary in agriculture, for example pre-emergence application, post-emergence application and seed dressing, and also various methods and techniques such as, for example, the controlled release of active ingredient. For that purpose a solution of the active ingredient is applied to mineral granule carriers or polymerised granules (urea/formaldehyde) and dried. If required, it is also possible to apply a coating (coated granules), which allows the active ingredient to be released in metered amounts over a specific period of time.

The compounds of formula I may be used as herbicides in their unmodified form, that is to say as obtained in the synthesis, but they are preferably formulated in customary manner together with the adjuvants conventionally employed in formulation technology, for example into emulsifiable concentrates, directly sprayable or dilutable solutions, dilute emulsions, wettable powders, soluble powders, dusts, granules or microcapsules. Such formulations are described, for example, on pages 9 to 13 of WO 97/34485. As with the nature of the compositions, the methods of application, such as spraying, atomising, dusting, wetting, scattering or pouring, are chosen in accordance with the intended objectives and the prevailing circumstances.

The formulations, that is to say the compositions, preparations or mixtures comprising the compound (active ingredient) of formula I or at least one compound of formula I and, usually, one or more solid or liquid formulation adjuvants, are prepared in known manner, e.g. by homogeneously mixing and/or grinding the active ingredients with the formulation adjuvants, for example solvents or solid carriers. Surface-active compounds (surfactants) may also be used in addition in the preparation of the formulations. Examples of solvents and solid carriers are given, for example, on page 6 of WO 97/34485.

Depending upon the nature of the compound of formula I to be formulated, suitable surface-active compounds are non-ionic, cationic and/or anionic surfactants and surfactant mixtures having good emulsifying, dispersing and wetting properties. Examples of suitable anionic, non-ionic and cationic surfactants are listed, for example, on pages 7 and 8 of WO 97/34485. In addition, the surfactants conventionally employed in formulation technology, which are described, inter alia, in “McCutcheon's Detergents and Emulsifiers Annual” MC Publishing Corp., Ridgewood N.J., 1981, Stache, H., “Tensid-Taschen-buch”, Carl Hanser Verlag, Munich/Vienna 1981, and M. and J. Ash, “Encyclopedia of Surfactants”, Vol. 1-Ill, Chemical Publishing Co., New York, 1980-81, are also suitable for the preparation of the herbicidal compositions according to the invention.

The herbicidal formulations generally contain from 0.1 to 99% by weight, especially from 0.1 to 95% by weight, of herbicide, from 1 to 99.9% by weight, especially from 5 to 99.8% by weight, of a solid or liquid formulation adjuvant, and from 0 to 25% by weight, especially from 0.1 to 25% by weight, of a surfactant. Whereas commercial products will preferably be formulated as concentrates, the end user will normally employ dilute formulations. The compositions may also comprise further ingredients, such as stabilisers, for example vegetable oils or epoxidised vegetable oils (epoxidised coconut oil, rapeseed oil or soybean oil), anti-foams, for example silicone oil, preservatives, viscosity regulators, binders, tackifiers, and also fertilisers or other active ingredients.

The compounds of formula I are generally applied to plants or the locus thereof at rates of application of from 0.001 to 4 kg/ha, especially from 0.005 to 2 kg/ha. The concentration required to achieve the desired effect can be determined by experiment. It is dependent on the nature of the action, the stage of development of the cultivated plant and of the weed and on the application (place, time, method) and may vary within wide limits as a function of those parameters.

The compounds of formula I are distinguished by herbicidal and growth-inhibiting properties, allowing them to be used in crops of useful plants, especially cereals, cotton, soybeans, sugar beet, sugar cane, plantation crops, rape, maize and rice, and also for non-selective weed control. The term “crops” is to be understood as including also crops that have been made tolerant to herbicides or classes of herbicides as a result of conventional methods of breeding or genetic techniques. The weeds to be controlled may be either monocotyledonous or dicotyledonous weeds, such as, for example, Stellaria, Nasturtium, Agrostis, Digitaria, Avena, Setaria, Sinapis, Lolium, Solanum, Echinochloa, Scirpus, Monochoria, Sagittaria, Bromus, Alopecurus, Sorghum halepense, Panicum, Rottboellia, Cyperus, Abutilon, Sida, Xanthium, Amaranthus, Chenopodium, Ipomoea, Chrysanthemum, Galium, Viola and Veronica.

The following Examples further illustrate but do not limit the invention.

### PREPARATION EXAMPLES

### EXAMPLE P1

### Preparation of 3-methoxy-4-prop-2-ynyloxy-benzaldehyde O-methyl-oxime

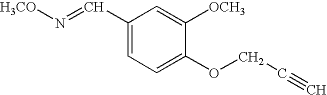

5.0 g (26.3 mmol) of 3-methoxy-4-(2-propynyloxy)-benzaldehyde (see DE-A-4 141 401) are dissolved at 20° C. in 20 ml of ethanol under nitrogen. Then, with stirring, 2.86 g (34.3 mmol) of O-methyl-hydroxylamine hydrochloride and 4.65 g (34.2 mmol) of anhydrous sodium acetate are added in succession thereto. After the addition, stirring is carried out for a further 18 hours at 20° C. and 1.5 hours at about 50° C. The solvent is then distilled off, 100 ml of water are added to the residue and extraction is carried out three times with a total of 100 ml of dichloromethane. The combined organic phases are dried over magnesium sulfate. After evaporating off the solvent, 5.37 g of the desired target compound 3-methoxy-4-prop-2-ynyloxy-benzaldehyde O-methyl-oxime are obtained in the form of yellow crystals having a melting point of 68-69° C.

¹H-NMR (CDCl₃): δ (ppm)=2.53 (t); 3.92 (s); 3.97 (s); 4.80 (t); 7.00 (s); 7.29 (s); 8.00 (s).

### Example P2

### Preparation of 4-fluoro-2-methoxy-1-prop-2-ynyloxy-benzene

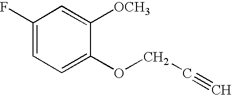

80.0 g (0.563 mol) of 4-fluoro-2-methoxyphenol are dissolved at 200C in 2 litres of acetone. 80.0 g of potassium carbonate are added and stirring is carried out at 200C for 1 hour. Then, in the course of 30 minutes, 82.7 ml of propargyl bromide are added dropwise, with stirirng, and the resulting suspension is heated at reflux temperature. When the reaction is complete, the solvent is distilled off and the residue is taken up in ether. The ether phase is washed three times with 1N NaOH, twice with water and twice with saturated brine. A small amount of toluene is then added to the ether phase and the reaction mixture is finally completely concentrated by evaporation. 171.6 g of the desired target compound 4-fluoro-2-methoxy-1-prop-2-ynyloxy-benzene are obtained in the form of a light-brown oil.

¹H-NMR (CDCl₃): δ (ppm)=2.52 (s); 3.86 (s); 4.72 (s); 6.58-6.72 (m); 6.95-7.05 (m).

### Example P3

### 2-Chloro-5-iodopyridine

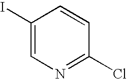

22.1 g (0.1 mol) of 2-hydroxy-5-iodo-pyridine are heated together with 31.0 g (0.2 mol) of phosphorus oxytrichloride (POCl₃) for 1 hour at reflux temperature. When the reaction is complete, excess POCl₃ is distilled off and the residue is taken up in toluene. The organic phase is stirred with aqueous potassium carbonate solution, separated and concentrated by evaporation. The crude product is purified by chromatography over silica gel. 19 g of the desired title compound are obtained in the form of colourless crystals.

¹H-NMR (CDCl₃): δ (ppm)=7.10-7.20 (d); 7.90-8.00 (dxd); 8.55-8.65 (d).

### Example P4

### 2-Chloro-5-[3-(4-fluoro-2-methoxy-phenoxy)-prop-1-ynyl]-pyridine

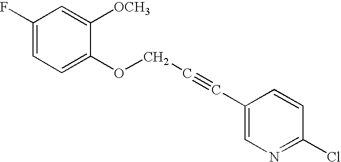

300 mg (1.25 mmol) of 2-chloro-5-iodo-pyridine (Example P3), 339 mg (1.87 mmol) of 4-fluoro-2-methoxy-1-prop-2-ynyloxy-benzene (Example P2) and 48 mg (0.25 mmol) of copper(I) iodide (CuI) are suspended in a mixture consisting of 4 ml of dioxane and 3 ml of diisopropylamine under argon at 20° C. The resulting reaction mixture is heated to 50° C. and 88 mg (0.125 mmol) of Pd(PPh₃)₂Cl₂ are added. After 3.5 hours, the reaction mixture is cooled to 20° C. The solvent mixture is distilled off in vacuo and the crude product is subjected to flash chromatography over silica gel (eluant: ethyl acetate/petroleum ether 1/5). 308 mg of the desired target compound 2-chloro-5-[3-(4-fluoro-2-methoxy-phenoxy)-prop-1-ynyl]-pyridine are obtained in the form of a beige solid having a melting point of 86-87° C.

¹H-NMR (CDCl₃): δ (ppm)=3.87 (s); 4.93 (s); 6.56-6.70 (m); 6.97-7.02 (dxd); 7.28 (d); 7.64 (dxd); 8.42 (d).

### Example P5

### 2-[3-(4-Fluoro-2-methoxy-phenoxy)-prop-1-ynyl]-5-methyl-pyridine

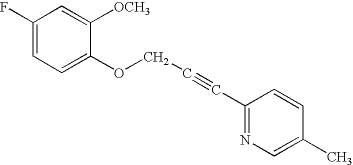

200 mg (1.16 mmol) of 2-bromo-5-methyl-pyridine, 314 mg (1.74 mmol) of 4-fluoro-2-methoxy-1-prop-2-ynyloxy-benzene (Example P2) and 44 mg (0.23 mmol) of copper(I) iodide (CuI) are suspended in a mixture consisting of 4 ml of dioxane and 3 ml of diisopropylamine under argon at 20° C. The reaction mixture is heated to 50° C. and 81 mg (0.12 mmol) of Pd(PPh₃)₂CO₂ are added. After 4 hours, the reaction mixture is cooled to 20° C. The solvent mixture is distilled off in vacuo and the resulting crude product is purified by chromatography over silica gel (eluant: ethyl acetate/petroleum ether 1/3). 208 mg of the desired target compound 2-[3-(4-fluoro-2-methoxy-phenoxy)-prop-1-ynyl]-5-methyl-pyridine are obtained in the form of a brown oil.

¹H-NMR (CDCl₃): δ (ppm)=2.33 (s); 3.86 (s); 4.95 (s); 6.55-6.68 (m); 7.05 (dxd); 7.29 (d); 7.43 (dxd); 8.40 (d).

### Example P6

### 2-[3-(4-Fluoro-2-methoxy-phenoxy)-prop-1-ynyl]-4-methyl-pyridine

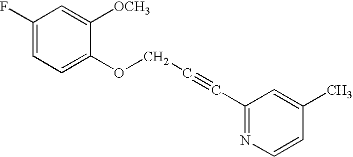

200 mg (1.16 mmol) of 2-bromo-4-methyl-pyridine, 314 mg (1.74 mmol) of 4-fluoro-2-methoxy-1-prop-2-ynyloxy-benzene (Example P2) and 44 mg (0.23 mmol) of copper(I) iodide (CuI) are suspended in a mixture consisting of 4 ml of dioxane and 3 ml of diisopropylamine under argon at 20° C. The reaction mixture is heated to 50° C. and 81 mg (0.12 mmol) of Pd(PPh₃)₂Cl₂ are added. After 4 hours, the reaction mixture is cooled to 20° C. The solvent mixture is distilled off in vacuo and the resulting crude product is purified by chromatography over silica gel (eluant: ethyl acetate/petroleum ether 1/3). 152 mg of the desired target compound 2-[3-(4-fluoro-2-methoxy-phenoxy)-prop-1-ynyl]-4-methyl-pyridine are obtained in the form of a brown solid.

¹H-NMR (CDCl₃): δ (ppm)=2.32 (s); 3.87 (s); 4.95 (s); 6.56-6.68 (m); 7.03-7.08 (m); 7.23 (s); 8.41 (d).

In a manner analogous to that described in Examples P1 to P5 or in accordance with the methods as shown in Reaction Schemes 1-5 and in the references indicated, it is also possible to obtain the preferred compounds listed in the following Tables. In the column headed “Phys. data”, the temperatures indicate the melting point (m.p.) of the compounds in question. In cases where the purity of the compounds has been investigated by means of HPLC/MS (“High Pressure Liquid Chromatography/Electrospray Mass Spectrometry”), the column headed “Phys. data” gives the [M+H]⁺ peak from the Electrospray-MS of the compound in question (e.g. Comp. No. 3.011).

TABLE 1
Compounds of formula I₁

| | |
|---|---|
| | (I₁) |
| 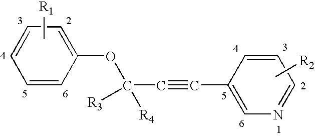 | |

| Comp. | | | | | Phys. data |
|---|---|---|---|---|---|
| No. | R₁ | R₂ | R₃ | R₄ | m.p. (° C.) |
| 1.001 | 2-OCH₃, 4-CN | 2-Cl | H | H | 160-161 |
| 1.002 | 2-F, 4-Cl | 2-Cl | H | H | |
| 1.003 | 2-Cl, 4-Cl | 2-Cl | H | H | |
| 1.004 | 2-OCH₃, 4-F | 2-Cl | H | H | 86-87 |
| 1.005 | 2-OCH₃, 4-Cl | 2-Cl | H | H | |
| 1.006 | 2-OCH₃, 4-Br | 2-Cl | H | H | |
| 1.007 | 2-CF₃, 4-F | 2-Cl | H | H | |
| 1.008 | 2-OCH₃, 4-CF₃ | 2-Cl | H | H | |
| 1.009 | 2-OCH₃, 4-CH₃ | 2-Cl | H | H | |
| 1.010 | 2-OCH₃, 4-CH═NOCH₃ | 2-Cl | H | H | 97-99 |
| 1.011 | 2-OCH₃, 5-CH═NOCH₃ | 2-Cl | H | H | 128-129 |
| 1.012 | 3-CF₃ | 2-OCH₂CH₂N(C₂H₅)₂ | H | H | oil |
| 1.013 | 4-OCH3 | 2-OCH₂CH₂N(C₂H₅)₂ | H | H | oil |
| 1.014 | H | 2-OCH₂CH₂N(C₂H₅)₂ | H | H | oil |
| 1.015 | 2-Cl | 2-OCH₂CH₂N(C₂H₅)₂ | H | H | oil |
| 1.016 | 4-Cl | 2-OCH₂CH₂N(C₂H₅)₂ | H | H | oil |
| 1.017 | 3-Cl | 2-OCH₂CH₂N(C₂H₅)₂ | H | H | oil |
| 1.018 | 2-OCH₃, 4-F | H | H | H | 78-79 |
| 1.019 | 2-OCH₃, 4-CN | 2-Cl | CH₃ | H | — |
| 1.020 | 2-F, 4-Cl | 2-Cl | CH₃ | H | — |
| 1.021 | 2-Cl, 4-Cl | 2-Cl | CH₃ | H | — |
| 1.022 | 2-OCH₃, 4-F | 2-Cl | CH₃ | H | — |
| 1.023 | 2-OCH₃, 4-Cl | 2-Cl | CH₃ | H | — |
| 1.024 | 2-OCH₃, 4-Br | 2-Cl | CH₃ | H | — |
| 1.025 | 2-CF₃, 4-F | 2-Cl | CH₃ | H | — |
| 1.026 | 2-OCH₃, 4-CF₃ | 2-Cl | CH₃ | H | — |
| 1.027 | 2-OCH₃, 4-CH₃ | 2-Cl | CH₃ | H | — |
| 1.028 | 2-OCH₃, 4-CH═NOCH₃ | 2-Cl | CH₃ | H | — |
| 1.029 | 2-OCH₃, 4-CH═NOCH₃ | 2-NH₂ | H | H | 135-138 |
| 1.030 | 2-OCH₃, 4-F | 2-NH₂ | H | H | — |
| 1.031 | 2-OCH₃, 4-Cl | 2-NH₂ | H | H | — |
| 1.032 | 2-OCH₃, 4-CN | 3-Br | H | H | — |
| 1.033 | 2-F, 4-Cl | 3-Br | H | H | — |
| 1.034 | 2-Cl, 4-Cl | 3-Br | H | H | — |
| 1.035 | 2-OCH₃, 4-F | 3-Br | H | H | 72-74 |
| 1.036 | 2-OCH₃, 4-Cl | 3-Br | H | H | — |
| 1.037 | 2-OCH₃, 4-Br | 3-Br | H | H | — |
| 1.038 | 2-CF₃, 4-F | 3-Br | H | H | — |
| 1.039 | 2-OCH₃, 4-CF₃ | 3-Br | H | H | — |
| 1.040 | 2-OCH₃, 4-OH₃ | 3-Br | H | H | — |
| 1.041 | 2-OCH₃, 4-CH═NOCH₃ | 3-Br | H | H | 102-104 |
| 1.042 | 2-OCH₃, 4-CH═NOCH₃ | 3-Br, 6-OH | H | H | crystalline |
| 1.043 | 2-OCH₃, 4-F | 3-Br, 6-OH | H | H | crystalline |
| 1.044 | 2-OCH₃, 4-CN | 3-CH₂CN | H | H | — |
| 1.045 | 2-F, 4-Cl | 3-CH₂CN | H | H | — |
| 1.046 | 2-Cl, 4-Cl | 3-CH₂CN | H | H | — |
| 1.047 | 2-OCH₃, 4-F | 3-CH₂CN | H | H | — |
| 1.048 | 2-OCH₃, 4-Cl | 3-CH₂CN | H | H | — |
| 1.049 | 2-OCH₃, 4-Br | 3-CH₂CN | H | H | — |
| 1.050 | 2-CF₃, 4-F | 3-CH₂CN | H | H | — |
| 1.051 | 2-OCH₃, 4-CF₃ | 3-CH₂CN | H | H | — |
| 1.052 | 2-OCH₃, 4-CH₃ | 3-CH₂CN | H | H | — |
| 1.053 | 2-OCH₃, 4-CH═NOCH₃ | 3-CH₂CN | H | H | — |
| 1.054 | 2-OCH₃, 4-F | 3-OCH₃, | H | H | crystalline |
| | | 6-NHC(O)O-t-C₄H₉ | | | |
| 1.055 | 2-OCH₃, 4-CH═NOCH₃ | 3-OCH₃, | H | H | crystalline |
| | | 6-NHC(O)O-t-C₄H₉ | | | |
| 1.056 | 2-OCH₃, 4-F | 3-OCH₃, 6-NH₂ | H | H | amorphous |
| 1.057 | 2-OCH₃, 4-CH═NOCH₃ | 3-OCH₃, 6-NH₂ | H | H | crystalline |
| 1.058 | 2-OCH₃, 4-CN | 3-Cl | H | H | — |
| 1.059 | 2-F, 4-Cl | 3-Cl | H | H | — |
| 1.060 | 2-Cl, 4-Cl | 3-Cl | H | H | — |
| 1.061 | 2-OCH₃, 4-F | 3-Cl | H | H | — |
| 1.062 | 2-OCH₃, 4-Cl | 3-Cl | H | H | — |
| 1.063 | 2-OCH₃, 4-Br | 3-Cl | H | H | — |
| 1.064 | 2-CF₃, 4-F | 3-Cl | H | H | — |
| 1.065 | 2-OCH₃, 4-CF₃ | 3-Cl | H | H | — |
| 1.066 | 2-OCH₃, 4-CH₃ | 3-Cl | H | H | — |
| 1.067 | 2-OCH₃, 4-CH═NOCH₃ | 3-Cl | H | H | — |
| 1.068 | 2-OCH₃, 4-F | 3-Cl, 6-OH | H | H | — |
| 1.069 | 2-OCH₃, 4-CH═NOCH₃ | 3-Cl, 6-OH | H | H | crystalline |
| 1.070 | 2-OCH₃, 4-CN | 3-CH(CH₃)CN | H | H | — |
| 1.071 | 2-F, 4-Cl | 3-CH(CH₃)CN | H | H | — |
| 1.072 | 2-Cl, 4-Cl | 3-CH(CH₃)CN | H | H | — |
| 1.073 | 2-OCH₃, 4-F | 3-CH(CH₃)CN | H | H | — |
| 1.074 | 2-OCH₃, 4-Cl | 3-CH(CH₃)CN | H | H | — |
| 1.075 | 2-OCH₃, 4-Br | 3-CH(CH₃)CN | H | H | — |
| 1.076 | 2-CF₃, 4-F | 3-CH(CH₃)CN | H | H | — |
| 1.077 | 2-OCH₃, 4-CF₃ | 3-CH(CH₃)CN | H | H | — |
| 1.078 | 2-OCH₃, 4-CH₃ | 3-CH(CH₃)CN | H | H | — |
| 1.079 | 2-OCH₃, 4-CH═NOCH₃ | 3-CH(CH₃)CN | H | H | — |
| 1.080 | 2-OCH₃, 4-F | 3-CH₂CN | CH₃ | CH₃ | — |
| 1.081 | 2-OCH₃, 4-Cl | 3-CH₂CN | CH₃ | CH₃ | — |
| 1.082 | 2-OCH₃, 4-Br | 3-CH₂CN | CH₃ | CH₃ | — |
| 1.083 | 2-OCH₃, 4-CN | 3-CH₃ | H | H | — |
| 1.084 | 2-F, 4-Cl | 3-CH₃ | H | H | — |
| 1.085 | 2-Cl, 4-Cl | 3-CH₃ | H | H | — |
| 1.086 | 2-OCH₃, 4-F | 3-CH₃ | H | H | — |
| 1.087 | 2-OCH₃, 4-Cl | 3-CH₃ | H | H | — |
| 1.088 | 2-OCH₃, 4-Br | 3-CH₃ | H | H | — |
| 1.089 | 2-CF₃, 4-F | 3-CH₃ | H | H | — |
| 1.090 | 2-OCH₃, 4-CF₃ | 3-CH₃ | H | H | — |
| 1.091 | 2-OCH₃, 4-CH₃ | 3-CH₃ | H | H | — |
| 1.092 | 2-OCH₃, 4-CH═NOCH₃ | 3-CH₃ | H | H | — |
| 1.093 | 2-OCH₃ | 3-CH₂CN | H | H | — |
| 1.094 | 2-OCH₃ | 4-CH₂CN | H | H | — |
| 1.095 | 2-OCH₃ | 3-F | H | H | — |
| 1.096 | 2-OCH₃ | 3-Cl | H | H | — |
| 1.097 | 2-OCH₃ | 3-Br | H | H | — |
| 1.098 | 2-OCH₃, 4-F | 2-OCH₃ | H | H | 66-68 |
| 1.099 | 2-OCH₃, 4-CH═NOCH₃ | 2-CH₃ | H | H | resin |
| 1.100 | 2-OCH₃, 4-F | 2-CH₃ | H | H | resin |
| 1.101 | 2-OCH₃, 4-CH═NOCH₃ | 2-CN | H | H | crystalline |
| 1.102 | 2-OCH₃, 4-CH═NOCH₃ | 3-OCH₃ | H | H | resin |
| 1.103 | 2-OCH₃, 4-F | 3-OCH₃ | H | H | resin |
| 1.104 | 2-OCH₃, 4-F | 2-CN | H | H | oil |

TABLE 2
Compounds of formula I₂:

| | |
|---|---|
| | (I₂) |
| 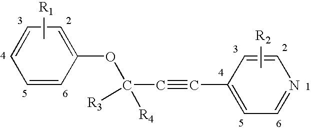 | |

| Comp. | | | | | Phys. data |
|---|---|---|---|---|---|
| No. | R₁ | R₂ | R₃ | R₄ | m.p. (° C.) |
| 2.001 | 2-OCH₃, 4-CN | 2-F | H | H | 132-134 |
| 2.002 | 2-F, 4-Cl | 2-F | H | H | — |
| 2.003 | 2-Cl, 4-Cl | 2-F | H | H | — |
| 2.004 | 2-OCH₃, 4-F | 2-F | H | H | resin |
| 2.005 | 2-OCH₃, 4-Cl | 2-F | H | H | — |
| 2.006 | 2-OCH₃, 4-Br | 2-F | H | H | — |
| 2.007 | 2-CF₃, 4-F | 2-F | H | H | — |
| 2.008 | 2-OCH₃, 4-CF₃ | 2-F | H | H | — |
| 2.009 | 2-OCH₃, 4-CH₃ | 2-F | H | H | — |
| 2.010 | 2-OCH₃,4-CH═NOCH₃ | 2-F | H | H | amorphous |
| 2.011 | 2-OCH₃, 4-F | H | H | H | crystalline |
| 2.012 | 2-OCH₃,4-CH═NOCH₃ | H | H | H | crystalline |
| 2.013 | 2-OCH₃, 4-CN | 2-OCH₃ | H | H | — |
| 2.014 | 2-F, 4-Cl | 2-OCH₃ | H | H | — |
| 2.015 | 2-Cl, 4-Cl | 2-OCH₃ | H | H | — |
| 2.016 | 2-OCH₃, 4-F | 2-OCH₃ | H | H | — |
| 2.017 | 2-OCH₃, 4-Cl | 2-OCH₃ | H | H | — |
| 2.018 | 2-OCH₃, 4-Br | 2-OCH₃ | H | H | — |
| 2.019 | 2-CF₃, 4-F | 2-OCH₃ | H | H | — |
| 2.020 | 2-OCH₃, 4-CF₃ | 2-OCH₃ | H | H | — |
| 2.021 | 2-OCH₃, 4-CH₃ | 2-OCH₃ | H | H | — |
| 2.022 | 2-OCH₃,4-CH═NOCH₃ | 2-OCH₃ | H | H | — |
| 2.023 | 2-OCH₃, 4-F | 2-OCH₃, 5-NH₂ | H | H | amorphous |
| 2.024 | 2-OCH₃,4-CH═NOCH₃ | 2-OCH₃, 5-NH₂ | H | H | amorphous |
| 2.025 | 2-OCH₃, 4-F | 2-OCH₃, | H | H | oil |
| | | 5-NHC(O)O-t-C₄H₉ | | | |
| 2.026 | 2-OCH₃,4-CH═NOCH₃ | 2-OCH₃, | H | H | crystalline |
| | | 5-NHO(O)O-t-C₄H₉ | | | |
| 2.027 | 2-OCH₃, 4-CN | 2-Cl | H | H | — |
| 2.028 | 2-F, 4-Cl | 2-Cl | H | H | — |
| 2.029 | 2-Cl, 4-Cl | 2-Cl | H | H | — |
| 2.030 | 2-OCH₃, 4-F | 2-Cl | H | H | — |
| 2.031 | 2-OCH₃, 4-Cl | 2-Cl | H | H | — |
| 2.032 | 2-OCH₃, 4-Br | 2-Cl | H | H | — |
| 2.033 | 2-CF₃, 4-F | 2-Cl | H | H | — |
| 2.034 | 2-OCH₃, 4-CF₃ | 2-Cl | H | H | — |
| 2.035 | 2-OCH₃, 4-CH₃ | 2-Cl | H | H | — |
| 2.036 | 2-OCH₃, 4-CH═NOCH₃ | 2-Cl | H | H | — |
| 2.037 | 2-OCH₃, 4-CN | 2-CH₂CN | H | H | — |
| 2.038 | 2-F, 4-Cl | 2-CH₂CN | H | H | — |
| 2.039 | 2-Cl, 4-Cl | 2-CH₂CN | H | H | — |
| 2.040 | 2-OCH₃, 4-F | 2-CH₂CN | H | H | 83-84 |
| 2.041 | 2-OCH₃, 4-Cl | 2-CH₂CN | H | H | — |
| 2.042 | 2-OCH₃, 4-Br | 2-CH₂CN | H | H | — |
| 2.043 | 2-CF₃, 4-F | 2-CH₂CN | H | H | — |
| 2.044 | 2-OCH₃, 4-CF₃ | 2-CH₂CN | H | H | — |
| 2.045 | 2-OCH₃, 4-CH₃ | 2-CH₂CN | H | H | — |
| 2.046 | 2-OCH₃, 4-CH═NOCH₃ | 2-CH₂CN | H | H | resin |
| 2.047 | 2-OCH₃, 4-CN | 2-N(CH₃)₂ | H | H | 142-144 |
| 2.048 | 2-F, 4-Cl | 2-N(CH₃)₂ | H | H | — |
| 2.049 | 2-Cl, 4-Cl | 2-N(CH₃)₂ | H | H | — |
| 2.050 | 2-OCH₃, 4-F | 2-N(CH₃)₂ | H | H | — |
| 2.051 | 2-OCH₃, 4-Cl | 2-N(CH₃)₂ | H | H | — |
| 2.052 | 2-OCH₃, 4-Br | 2-N(CH₃)₂ | H | H | — |
| 2.053 | 2-CF₃, 4-F | 2-N(CH₃)₂ | H | H | — |
| 2.054 | 2-OCH₃, 4-CF₃ | 2-N(CH₃)₂ | H | H | — |
| 2.055 | 2-OCH₃, 4-CH₃ | 2-N(CH₃)₂ | H | H | — |
| 2.056 | 2-OCH₃, 4-CH═NOCH₃ | 2-N(CH₃)₂ | H | H | — |
| 2.057 | 2-OCH₃, 4-CN | 2-CH(CH₃)CN | H | H | — |
| 2.058 | 2-F, 4-Cl | 2-CH(CH₃)CN | H | H | — |
| 2.059 | 2-Cl, 4-Cl | 2-CH(CH₃)CN | H | H | — |
| 2.060 | 2-OCH₃, 4-F | 2-CH(CH₃)CN | H | H | — |
| 2.061 | 2-OCH₃, 4-Cl | 2-CH(CH₃)CN | H | H | — |
| 2.062 | 2-OCH₃, 4-Br | 2-CH(CH₃)CN | H | H | — |
| 2.063 | 2-CF₃, 4-F | 2-CH(CH₃)CN | H | H | — |
| 2.064 | 2-OCH₃, 4-CF₃ | 2-CH(CH₃)CN | H | H | — |
| 2.065 | 2-OCH₃, 4-CH₃ | 2-CH(CH₃)CN | H | H | — |
| 2.066 | 2-OCH₃, 4-CH═NOCH₃ | 2-CH(CH₃)CN | H | H | — |
| 2.067 | 2-OCH₃, 4-F | 2-Cl | CH₃ | H | — |
| 2.068 | 2-OCH₃, 4-Cl | 2-Cl | CH₃ | H | — |
| 2.069 | 2-OCH₃, 4-Br | 2-Cl | CH₃ | H | — |
| 2.070 | 2-OCH₃, 4-CF₃ | 2-Cl | CH₃ | H | — |
| 2.071 | 2-OCH₃, 4-CH═NOCH₃ | 2-Cl | CH₃ | H | — |
| 2.072 | 2-OCH₃, 4-F | 2-CH₂CN | CH₃ | CH₃ | — |
| 2.073 | 2-OCH₃, 4-Cl | 2-CH₂CN | CH₃ | CH₃ | — |
| 2.074 | 2-OCH₃, 4-Br | 2-CH₂CN | CH₃ | CH₃ | — |
| 2.075 | 2-OCH₃, 4-CF₃ | 2-CH₂CN | CH₃ | CH₃ | — |
| 2.076 | 2-OCH₃, 4-CH═NOCH₃ | 2-CH₂CN | CH₃ | CH₃ | — |
| 2.077 | 2-OCH₃, 4-F | 2-CH₂CN | CH₃ | H | — |
| 2.078 | 2-OCH₃, 4-Cl | 2-CH₂CN | CH₃ | H | — |
| 2.079 | 2-OCH₃, 4-Br | 2-CH₂CN | CH₃ | H | — |
| 2.080 | 2-OCH₃, 4-CF₃ | 2-CH₂CN | CH₃ | H | — |
| 2.081 | 2-OCH₃, 4-CH═NOCH₃ | 2-CH₂CN | CH₃ | H | — |
| 2.082 | 2-OCH₃, 4-F | 3-CH₂CN | CH₃ | H | — |
| 2.083 | 2-OCH₃, 4-Cl | 3-CH₂CN | CH₃ | H | — |
| 2.084 | 2-OCH₃, 4-Br | 3-CH₂CN | CH₃ | H | — |
| 2.085 | 2-OCH₃, 4-CF₃ | 3-CH₂CN | CH₃ | H | — |
| 2.086 | 2-OCH₃, 4-CH═NOCH₃ | 3-CH₂CN | CH₃ | H | — |
| 2.087 | 2-OCH₃ | 2-CH₂CN | H | H | — |
| 2.088 | 2-OCH₃ | 3-CH₂CN | H | H | — |
| 2.089 | 2-OCH₃ | 2-F | H | H | — |
| 2.090 | 2-OCH₃ | 2-Cl | H | H | — |
| 2.091 | 2-OCH₃ | 2-Br | H | H | — |

TABLE 3
Compounds of formula I₃:

| | |
|---|---|
| | (I₃) |
| 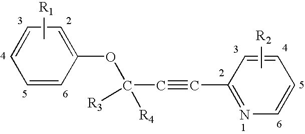 | |

| Comp. | | | | | Phys. data |
|---|---|---|---|---|---|
| No. | R₁ | R₂ | R₃ | R₄ | m.p. (° C.) |
| 3.001 | 2-OCH₃, 4-CN | 4-CH₃ | H | H | — |
| 3.002 | 2-F, 4-Cl | 4-CH₃ | H | H | — |
| 3.003 | 2-Cl, 4-Cl | 4-CH₃ | H | H | — |
| 3.004 | 2-OCH₃, 4-F | 4-CH₃ | H | H | crystalline |
| 3.005 | 2-OCH₃, 4-Cl | 4-CH₃ | H | H | — |
| 3.006 | 2-OCH₃, 4-Br | 4-CH₃ | H | H | — |
| 3.007 | 2-CF₃, 4-F | 4-CH₃ | H | H | — |
| 3.008 | 2-OCH₃, 4-CF₃ | 4-CH₃ | H | H | — |
| 3.009 | 2-OCH₃, 4-CH₃ | 4-CH₃ | H | H | — |
| 3.010 | 2-OCH₃, 4-CH═NOCH₃ | 4-CH₃ | H | H | |
| 3.011 | H | 4-CH₃ | H | H | MS: [M+H]⁺ |
| 3.012 | 2-OCH₃, 4-OH2ON | 4-CH₃ | H | H | MS: [M+H]⁺ |
| 3.013 | 4-NO₂ | 3-OH, 6-CH₃ | H | H | MS: [M+H]⁺ |
| 3.014 | 2-OCH₃ | 3-OH, 6-CH₃ | H | H | MS: [M+H]⁺ |
| 3.015 | 4-CH₂CN | 3-OH, 6-CH₃ | H | H | MS: [M+H]⁺ |
| 3.016 | 2-OCH₃, 4-CH₂CN | 3-OH, 6-CH₃ | H | H | MS: [M+H]⁺ |
| 3.017 | 4-CN | 3-OH, 6-CH₃ | H | H | MS: [M+H]⁺ |
| 3.018 | 4-CO₂C₂H₅ | 3-OH, 6-CH₃ | H | H | MS: [M+H]⁺ |
| 3.019 | 2-Cl, 6-Cl | 3-OH, 6-CH₃ | H | H | MS: [M+H]⁺ |
| 3.020 | H | 3-OH, 6-CH₃ | H | H | MS: [M+H]⁺ |
| 3.021 | 2-OCH₃, 4-F | 6-CH₃ | H | H | oil |
| 3.022 | 2-OCH₃, 4-F | 5-CH₃ | H | H | oil |
| 3.023 | 2-OCH₃, 4-CH═NOCH₃ | 5-CH₃ | H | H | crystalline |
| 3.024 | 2-OCH₃, 4-CH═NOCH₃ | 6-CH₃ | H | H | crystalline |
| 3.025 | 4-OC₆H₅ | H | H | H | — |
| 3.026 | 2-OCH₃, 4-CH₂CN | H | H | H | MS: [M+H]⁺ |
| 3.027 | 4-CH₂CN | H | H | H | MS: [M+H]⁺ |
| 3.028 | H | H | H | H | MS: [M+H]⁺ |
| 3.029 | 2-OCH₃, 4-CN | 5-CF₃ | H | H | 94-95 |
| 3.030 | 2-F, 4-Cl | 5-CF₃ | H | H | — |
| 3.031 | 2-OCH₃, 4-F | 5-CF₃ | H | H | crystalline |
| 3.032 | 2-OCH₃, 4-Cl | 5-CF₃ | H | H | — |
| 3.033 | 2-OCH₃, 4-Br | 5-CF₃ | H | H | — |
| 3.034 | 2-OCH₃, 4-CF₃ | 5-CF₃ | H | H | — |
| 3.035 | 2-OCH₃, 4-CH₃ | 5-CF₃ | H | H | — |
| 3.036 | 2-OCH₃, 4-CH═NOCH₃ | 5-CF₃ | H | H | crystalline |
| 3.037 | 4-CO₂C₂H₅ | 5-CF₃ | H | H | MS: [M+H]⁺ |
| 3.038 | 2-OCH₃, 4-CN | 4-CH₂CN | H | H | — |
| 3.039 | 2-F, 4-Cl | 4-CH₂CN | H | H | — |
| 3.040 | 2-Cl, 4-Cl | 4-CH₂CN | H | H | — |
| 3.041 | 2-OCH₃, 4-F | 4-CH₂CN | H | H | — |
| 3.042 | 2-OCH₃, 4-Cl | 4-CH₂CN | H | H | — |
| 3.043 | 2-OCH₃, 4-Br | 4-CH₂CN | H | H | — |
| 3.044 | 2-CF₃, 4-F | 4-CH₂CN | H | H | — |
| 3.045 | 2-OCH₃, 4-CF₃ | 4-CH₂CN | H | H | — |
| 3.046 | 2-OCH₃, 4-CH₃ | 4-CH₂CN | H | H | — |
| 3.047 | 2-OCH₃, 4-CH═NOCH₃ | 4-CH₂CN | H | H | — |
| 3.048 | 2-OCH₃ | 4-CH₂CN | H | H | — |
| 3.049 | 2-OCH₃ | 4-Cl | H | H | — |
| 3.050 | 2-OCH₃ | 4-Br | H | H | — |
| 3.051 | 2-OCH₃ | 6-CH₂CN | H | H | 106 |
| 3.052 | 2-OCH₃ | 6-Cl | H | H | — |
| 3.053 | 2-OCH₃ | 6-Br | H | H | — |
| 3.054 | 2-OCH₃, 4-CN | 5-Cl | H | H | — |
| 3.055 | 2-F, 4-Cl | 5-Cl | H | H | — |
| 3.056 | 2-OCH₃, 4-F | 5-Cl | H | H | — |
| 3.057 | 2-OCH₃, 4-Cl | 5-Cl | H | H | — |
| 3.058 | 2-OCH₃, 4-Br | 5-Cl | H | H | — |
| 3.059 | 2-OCH₃, 4-CF₃ | 5-Cl | H | H | — |
| 3.060 | 2-OCH₃, 4-CH₃ | 5-Cl | H | H | — |
| 3.061 | 2-OCH₃, 4-CH═NOCH₃ | 5-Cl | H | H | — |
| 3.062 | 4-OCH₂CH₂N(C₂H₅)₂ | 5-Cl | H | H | 58-60 |
| 3.063 | 2-OCH₃, 4-CN | 6-Br | H | H | 84-85 |
| 3.064 | 2-F, 4-Cl | 6-Br | H | H | — |
| 3.065 | 2-Cl, 4-Cl | 6-Br | H | H | — |
| 3.066 | 2-OCH₃, 4-F | 6-Br | H | H | crystalline |
| 3.067 | 2-OCH₃, 4-Cl | 6-Br | H | H | — |
| 3.068 | 2-OCH₃, 4-Br | 6-Br | H | H | — |
| 3.069 | 2-CF₃, 4-F | 6-Br | H | H | — |
| 3.070 | 2-OCH₃, 4-CF₃ | 6-Br | H | H | — |
| 3.071 | 2-OCH₃, 4-CH₃ | 6-Br | H | H | — |
| 3.072 | 2-OCH₃, 4-CH═NOCH₃ | 6-Br | H | H | crystalline |
| 3.073 | 2-OCH₃, 4-F | 4-CH₃ | CH₃ | H | — |
| 3.074 | 2-OCH₃, 4-Cl | 4-CH₃ | CH₃ | H | — |
| 3.075 | 2-OCH₃, 4-Br | 4-CH₃ | CH₃ | H | — |
| 3.076 | 2-OCH₃, 4-CF₃ | 4-CH₃ | CH₃ | H | — |
| 3.077 | 2-OCH₃, 4-CH₃ | 4-CH₃ | CH₃ | H | — |
| 3.078 | 2-OCH₃, 4-CH═NOCH₃ | 4-CH₃ | CH₃ | H | — |
| 3.079 | 2-OCH₃, 4-F | 4-CH₃ | CH₃ | CH₃ | — |
| 3.080 | 2-OCH₃, 4-Cl | 4-CH₃ | CH₃ | CH₃ | — |
| 3.081 | 2-OCH₃, 4-Br | 4-CH₃ | CH₃ | CH₃ | — |
| 3.082 | 2-OCH₃, 4-CF₃ | 4-CH₃ | CH₃ | CH₃ | — |
| 3.083 | 2-OCH₃, 4-CH₃ | 4-CH₃ | CH₃ | CH₃ | — |
| 3.084 | 2-OCH₃, 4-CH═NOCH₃ | 4-CH₃ | CH₃ | CH₃ | — |
| 3.085 | 2-OCH₃, 4-F | 3-OH | H | H | crystalline |
| 3.086 | 2-OCH₃, 4-Cl | 3-OH | H | H | — |
| 3.087 | 2-OCH₃, 4-Br | 3-OH | H | H | — |
| 3.088 | 2-OCH₃, 4-CF₃ | 3-OH | H | H | — |
| 3.089 | 2-OCH₃, 4-CH₃ | 3-OH | H | H | — |
| 3.090 | 2-OCH₃, 4-CH═NOCH₃ | 3-OH | H | H | crystalline |
| 3.091 | 4-CH₂CN | 3-OC₂H₅ | H | H | MS: [M+H]⁺ |
| 3.092 | 2-OCH₃ | 3-OC₂H₅ | H | H | MS: [M+H]⁺ |
| 3.093 | 2-OCH₃, 4-CH₂CN | 3-OC₂H₅ | H | H | MS: [M+H]⁺ |
| 3.094 | 2-OCH₃, 4-CN | 3-OC₂H₅ | H | H | MS: [M+H]⁺ |
| 3.095 | 2-OCH₃, 4-F | 6-CH₂CN | H | H | resin |
| 3.096 | 2-OCH₃, 4-CH═NOCH₃ | 6-CH₂CN | H | H | solid |
| 3.097 | 2-OCH₃, 4-CH═NOCH₃ | 5-CH₂CN | H | H | crystalline |
| 3.098 | 2-OCH₃, 4-F | 5-CH₂CN | H | H | resin |
| 3.099 | 2-OCH₃, 4-CH═NOCH₃ | 6-OCH₃ | H | H | resin |
| 3.100 | 2-OCH₃, 4-F | 6-OCH₃ | H | H | resin |
| 3.101 | 2-OCH₃, 4-CH═NOCH₃ | H | H | H | resin |
| 3.102 | 2-OCH₃, 4-F | H | H | H | oil |

### Biological Examples

### Example B1: Herbicidal Action Prior to Emergence of the Plants (Pre-Emergence Action)

Monocotyledonous and dicotyledonous test plants are sown in standard soil in pots. Immediately after sowing, the test compounds, in the form of an aqueous suspension (prepared from a wettable powder (Example F3, b) according to WO 97/34485) or in the form of an emulsion (prepared from an emulsifiable concentrate (Example F1, c) according to WO 97/34485), are applied by spraying in an optimum concentration (500 litres of water/ha). The test plants are then grown in a greenhouse under optimum conditions. After a test duration of 4 weeks, the test is evaluated in accordance with a scale of nine ratings (1=total damage, 9=no action). Ratings of from 1 to 4 (especially from 1 to 3) indicate good to very good herbicidal action.

Test plants: Panicum, Echinochloa (Ds), Amaranthus, Chenopodium, Stellaria, Veronica.

TABLE B1
Concentration 1000 g of active ingredient/ha

| Comp. | Pani- | Echinochloa | Amaran- | Cheno- | | |
|---|---|---|---|---|---|---|
| No. | cum | (Ds) | thus | podium | Stellaria | Veronica |
| 1.010 | 3 | — | 1 | 1 | 1 | 1 |
| 1.004 | 2 | 2 | 1 | 1 | 1 | 1 |
| 3.004 | 2 | 2 | 1 | 1 | 1 | 1 |

The same results are obtained when the compounds of formula I are formulated in accordance with the other Examples analogously to WO 97/34485.

### Example B2

### Post-Emergence Herbicidal Action

Monocotyledonous and dicotyledonous test plants are sown in standard soil in pots. When the test plants are at the 2- to 3-leaf stage, the test compounds, in the form of an aqueous suspension (prepared from a wettable powder (Example F3, b) according to WO 97/34485) or in the form of an emulsion (prepared from an emulsifiable concentrate (Example F1, c) according to WO 97/34485), are applied by spraying in an optimum concentration (500 litres of water/ha). The test plants are then grown on in a greenhouse under optimum conditions. After a test duration of 2 to 3 weeks, the test is evaluated in accordance with a scale of nine ratings (1=total damage, 9=no action). Ratings of from 1 to 4 (especially from 1 to 3) indicate good to very good herbicidal action.

Test plants: Panicum, Euphorbia, Amaranthus, Chenopodium, Stellaria, Veronica.

TABLE B2
Concentration 1000 g of active ingredient/ha

| Comp. | Pani- | Euphor- | | Cheno- | | |
|---|---|---|---|---|---|---|
| No. | cum | bia | Amaranthus | podium | Stellaria | Veronica |
| 1.010 | 4 | 1 | 1 | 1 | 2 | 3 |
| 1.004 | — | 2 | 1 | 1 | 2 | 2 |
| 3.004 | 5 | 3 | 1 | 1 | 2 | 3 |

In the above Tables B1 and B2 “—” means that no data are available for that indication. The same results are obtained when the compounds of formula I are formulated in accordance with the other Examples analogously to WO 97/34485.

1. A compound of formula I 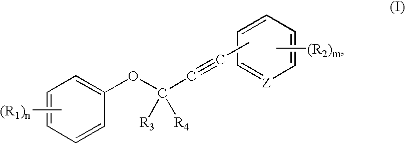
wherein
Z is ═N— or 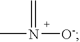
n is 0, 1, 2, 3, 4 or 5;
each R₁ independently of any others is halogen, —CN, —SCN, —SF₅, —NO₂, —NR₅R₆, —CO₂R₇, —CONR₈R₉, —C(R10)═NOR₁₁, —COR₁₂, —OR₁₃, —SR₁₄, —SOR₁₅, —SO₂R₁₆, —OSO₂R₁₇, C₁-C₈alkyl, C₂-C₈alkenyl, C₂-C₈alkynyl or C₃-C₆cycloalkyl; or is C₁-C₈alkyl, C₂-C₈alkenyl or C₂-C₈alkynyl substituted by one or more halogen, —CN, —NO₂, —NR₁₈R₁₉, —CO₂R₂₀, —CONR₂₁R₂₂, —COR₂₃, —C(R₂₄)═NOR₂₅, —C(S)NR₂₆R₂₇, —C(C₁-C₄alkylthio)═NR₂₈, —OR₂₉, —SR₃₀, —SOR₃₁, —SO₂R₃₂ or C₃-C₆cycloalkyl substituents; or
each R₁ independently of any others is C₃-C₆cycloalkyl substituted by one or more halogen, —CN, —NO₂, —NR₁₈R₁₉, —CO₂R₂₀, —CONR₂₁R₂₂, —COR₂₃, —C(R₂₄)═NOR₂₅, —C(S)NR₂₆R₂₇, —C(C₁-C₄alkylthio)═NR₂₈, —SR₃₀, —SOR₃₁, —SO₂R₃₂ or C₃-C₆cycloalkyl substituents; or
each R₁ independently of any others is phenyl, which may in turn be substituted by one or more halogen, C₁-C₄alkyl, C₁-C₄haloalkyl, C₁-C₄alkoxy, —CN, —NO₂, C₁-C₄alkylthio, C₁-C₄alkylsulfinyl or C₁-C₄alkylsulfonyl substituents; or
two adjacent R₁ together form a C₁-C₇alkylene bridge, which may be interrupted by 1 or 2 non-adjacent oxygen atoms and may be substituted by C₁-C₆alkyl or C₁-C₆alkoxy, the total number of ring atoms being at least 5 and at most 9; or
two adjacent R₁ together form a C₂-C₇alkenylene bridge, which may be interrupted by 1 or 2 non-adjacent oxygen atoms and may be substituted by C₁-C₆alkyl or C₁-C₆alkoxy, the total number of ring atoms being at least 5 and at most 9;
R₃ and R₄ are each independently of the other hydrogen, halogen, —CN, C₁-C₄alkyl or C₁-C₄alkoxy; or
R₃ and R₄ together are C₂-C₅alkylene;
R₅ is hydrogen or C₁-C₈alkyl;
R₆ is hydrogen, C₁-C₈alkyl, C₃-C₈alkenyl, C₃-C₈alkynyl, phenyl or benzyl; wherein phenyl and benzyl may in turn be substituted by one or more halogen, C₁-C₄alkyl, C₁-C₄haloalkyl, C₁-C₄alkoxy, —CN, —NO₂, C₁-C₄alkylthio, C₁-C₄alkylsulfinyl or C₁-C₄alkylsulfonyl substituents; or
R₅ and R₆ together are a C₂-C₅alkylene chain, which may be interrupted by an oxygen or a sulfur atom;
R₇ is hydrogen, C₁-C₈alkyl, C₃-C₈alkenyl or C₃-C₈alkynyl, or is C₁-C₈alkyl, C₃-C₈alkenyl or C₃-C₈alkynyl substituted by one or more halogen, C₁-C₄alkoxy or phenyl substituents, wherein phenyl may in turn be substituted by one or more halogen, C₁-C₄alkyl, C₁-C₄haloalkyl, C₁-C₄alkoxy, —CN, —NO₂, C₁-C₄alkylthio, C₁-C₄alkylsulfinyl or C₁-C₄alkylsulfonyl substituents;
R₈ is hydrogen or C₁-C₈alkyl;
R₉ is hydrogen or C₁-C₈alkyl, or is C₁-C₈alkyl substituted by one or more —COOH, C₁-C₈alkoxycarbonyl or —CN substituents, or
R₉ is C₃-C₈alkenyl, C₃-C₈alkynyl, phenyl or benzyl, wherein phenyl and benzyl may in turn be substituted by one or more halogen, C₁-C₄alkyl, C₁-C₄haloalkyl, C₁-C₄alkoxy, —CN, —NO₂, C₁-C₄alkylthio, C₁-C₄alkylsulfinyl or C₁-C₄alkylsulfonyl substituents; or
R₈ and R₉ together are C₂-C₅alkylene;
R₁₀ is hydrogen, C₁-C₄alkyl, C₁-C₄haloalkyl or C₃-C₆cycloalkyl;
R₁₁ is hydrogen, C₁-C₈alkyl, C₃-C₈alkenyl, C₃-C₈alkynyl, C₁-C₄haloalkyl or C₃-C₆haloalkenyl;
R₁₂ is hydrogen, C₁-C₄alkyl, C₁-C₄haloalkyl or C₃-C₆cycloalkyl;
R₁₃ is hydrogen, C₁-C₈alkyl, C₃-C₈alkenyl or C₃-C₈alkynyl; or
R₁₃ is phenyl or phenyl-C₁-C₆alkyl, wherein both phenyl rings may in turn be substituted by one or more halogen, C₁-C₄alkyl, C₁-C₄haloalkyl, C₁-C₄alkoxy, —CN, —NO₂, C₁-C₈alkylthio, C₁-C₈alkylsulfinyl or C₁-C₈alkylsulfonyl substituents, or
R₁₃ is C₁-C₈alkyl substituted by one or more halogen, —CN, C₁-C₆alkylamino, di(C₁-C₆alkyl)amino or C₁-C₄alkoxy substituents;
R₁₄ is hydrogen, C₁-C₈alkyl, C₃-C₈alkenyl or C₃-C₈alkynyl, or is C₁-C₈alkyl substituted by one or more halogen, —CN or C₁-C₄alkoxy substituents;
R₁₅, R₁₆ and R₁₇ are each independently of the others C₁-C₈alkyl, C₃-C₈alkenyl or C₃-C₈alkynyl, or C₁-C₈alkyl substituted by one or more halogen, —CN or C₁-C₄alkoxy substituents;
R₁₈ is hydrogen or C₁-C₈alkyl;
R₁₉ is hydrogen, C₁-C₈alkyl, C₃-C₈alkenyl, C₃-C₈alkynyl, phenyl or benzyl, wherein phenyl and benzyl may in turn be substituted by one or more halogen, C₁-C₄alkyl, C₁-C₄haloalkyl, C₁-C₄alkoxy, —CN, —NO₂, C₁-C₄alkylthio, C₁-C₄alkylsulfinyl or C₁-C₄alkylsulfonyl substituents; or
R₁₈ and R₁₉ together are a C₂-C₅alkylene chain, which may be interrupted by an oxygen or a sulfur atom;
R₂₀ is hydrogen, C₁-C₈alkyl, C₃-C₈alkenyl, C₃-C₈alkynyl, phenyl or benzyl, wherein phenyl and benzyl may in turn be substituted by one or more halogen, C₁-C₄alkyl, C₁-C₄haloalkyl, C₁-C₄alkoxy, —CN, —NO₂, C₁-C₄alkylthio, C₁-C₄alkylsulfinyl or C₁-C₄alkylsulfonyl substituents;
R₂₁ is hydrogen or C₁-C₈alkyl;
R₂₂ is hydrogen or C₁-C₈alkyl, or is C₁-C₈alkyl substituted by one or more —COOH, C₁-C₈-alkoxycarbonyl or —CN substituents, or
R₂₂ is C₃-C₈alkenyl, C₃-C₈alkynyl, phenyl or benzyl, wherein phenyl and benzyl may in turn be substituted by one or more halogen, C₁-C₄alkyl, C₁-C₄haloalkyl, C₁-C₄alkoxy, —CN, —NO₂, C₁-C₄alkylthio, C₁-C₄alkylsulfinyl or C₁-C₄alkylsulfonyl substituents; or
R₂₁ and R₂₂ together are C₂-C₅alkylene;
R₂₃ is hydrogen, C₁-C₄alkyl, C₁-C₄haloalkyl or C₃-C₆cycloalkyl;
R₂₄ is hydrogen, C₁-C₄alkyl, C₁-C₄haloalkyl or C₃-C₆cycloalkyl;
R₂₅ is hydrogen, C₁-C₈alkyl, C₃-C₈alkenyl, C₃-C₈alkynyl, C₁-C₄haloalkyl or C₃-C₆haloalkenyl;
R₂₆ is hydrogen or C₁-C₈alkyl;
R₂₇ is hydrogen or C₁-C₈alkyl, or is C₁-C₈alkyl substituted by one or more —COOH, C₁-C₈-alkoxycarbonyl or —CN substituents, or
R₂₇ is C₃-C₈alkenyl, C₃-C₈alkynyl, phenyl or benzyl, wherein phenyl and benzyl may in turn be substituted by one or more halogen, C₁-C₄alkyl, C₁-C₄haloalkyl, C₁-C₄alkoxy, —CN, —NO₂, C₁-C₄alkylthio, C₁-C₄alkylsulfinyl or C₁-C₄alkylsulfonyl substituents; or
R₂₆ and R₂₇ together are C₂-C₅alkylene;
R₂₈ is hydrogen or C₁-C₈alkyl;
R₂₉ and R₃₀ are each independently of the other hydrogen, C₁-C₈alkyl, C₃-C₈alkenyl or C₃-C₈alkynyl, or C₁-C₈alkyl substituted by one or more halogen, —CN or C₁-C₄alkoxy substituents;
R₃₁ and R₃₂ are each independently of the other C₁-C₈alkyl, C₃-C₈alkenyl or C₃-C₈alkynyl, or C₁-C₈alkyl substituted by one or more halogen, —CN or C₁-C₄alkoxy substituents;
m is 0, 1, 2, 3 or 4;
each R₂ independently of any others is halogen, —CN, —SCN, —OCN, —N₃, —SF₅, —NO₂, —NR₃₃R34, —CO₂R₃₅, —CONR₃₆R₃₇, —C(R₃₈)═NOR₃₉, —COR₄₀, —OR₄₁, —SR₄₂, —SOR₄₃, —SO₂R₄₄, —OSO₂R45, —N([CO]ₚR₄₆)COR₄₇, —N(OR₅₄)COR₅₅, —N(R₅₆)SO₂R₅₇, —N(SO₂R₅₈)SO₂R₅₉, —N═C(OR₆₀)R₆₁, —CR₆₂(OR₆₃)OR₆₄, —OC(O)NR₆₅R₆₆, —SC(O)NR₆₇R₆₈, —OC(S)NR₆₉R₇₀ or —N-phthalimide; or
R₂ is a 5- to 7-membered heterocyclic ring system which may be aromatic or partially or fully saturated and may contain from 1 to 4 hetero atoms selected from nitrogen, oxygen and sulfur, it being possible for that heterocyclic ring system in turn to be substituted by one or more halogen, C₁-C₄alkyl, C₁-C₄haloalkyl, hydroxy-C₁-C₄alkyl, C₁-C₄alkoxy, C₁-C₄alkoxy-C₁-C₄alkyl, —CN, —NO₂, C₁-C₆alkylthio, C₁-C₆alkylsulfinyl or C₁-C₆alkylsulfonyl substituents;
R₃₃ is hydrogen or C₁-C₈alkyl; and
R₃₄ is hydrogen, C₁-C₈alkyl, C₃-C₈alkenyl, C₃-C₈alkynyl, phenyl or benzyl, wherein phenyl and benzyl may in turn be substituted by one or more halogen, C₁-C₄alkyl, C₁-C₄haloalkyl, C₁-C₄alkoxy, —CN, —NO₂, C₁-C₄alkylthio, C₁-C₄alkylsulfinyl or C₁-C₄alkylsulfonyl substituents; or
R₃₃ and R34 together are a C₂-C₅alkylene chain, which may be interrupted by an oxygen or a sulfur atom;
R₃₅ is hydrogen, C₁-C₈alkyl, C₃-C₈alkenyl or C₃-C₈alkynyl, or is C₁-C₈alkyl, C₃-C₈alkenyl or C₃-C₈alkynyl substituted by one or more halogen, C₁-C₄alkoxy or phenyl substituents, wherein phenyl may in turn be substituted by one or more halogen, C₁-C₄alkyl, C₁-C₄haloalkyl, C₁-C₄alkoxy, —CN, —NO₂, C₁-C₄alkylthio, C₁-C₄alkylsulfinyl or C₁-C₄alkylsulfonyl substituents;
R₃₆ is hydrogen or C₁-C₈alkyl;
R₃₇ is hydrogen or C₁-C₈alkyl, or is C₁-C₈alkyl substituted by one or more —COOH, C₁-C₈-alkoxycarbonyl or —CN substituents, or
R₃₇ is C₃-C₈alkenyl, C₃-C₈alkynyl, phenyl or benzyl, wherein phenyl and benzyl may in turn be substituted by one or more halogen, C₁-C₄alkyl, C₁-C₄haloalkyl, C₁-C₄alkoxy, —CN, —NO₂, C₁-C₄alkylthio, C₁-C₄alkylsulfinyl or C₁-C₄alkylsulfonyl substituents; or
R₃₆ and R₃₇ together are C₃-C₅alkylene;
R38 is hydrogen, C₁-C₄alkyl, C₁-C₄haloalkyl or C₃-C₆cycloalkyl;
R₃₉ is hydrogen, C₁-C₈alkyl, C₃-C₈alkenyl, C₃-C₈alkynyl, C₁-C₄haloalkyl or C₃-C₆haloalkenyl;
R₄₀ is hydrogen, C₁-C₄alkyl, C₁-C₄haloalkyl, C₁-C₈alkylthio, —C(O)—C(O)OC₁-C₄alkyl or C₃-C₆-cycloalkyl;
R₄₁ is hydrogen, C₁-C₈alkyl, C₃-C₈alkenyl, C₃-C₈alkynyl, C₁-C₆alkoxy-C₁-C₆alkyl, C₁-C₈alkylcarbonyl, C₁-C₈alkoxycarbonyl, C₃-C₈alkenyloxycarbonyl, C₁-C₆alkoxy-C₁-C₆alkoxycarbonyl, C₁-C₆alkylthio-C₁-C₆alkyl, C₁-C₆alkylsulfinyl-C₁-C₆alkyl or C₁-C₆alkylsulfonyl-C₁-C₆alkyl; or
R₄₁ is phenyl or phenyl-C₁-C₆alkyl, wherein both phenyl rings may in turn be substituted by one or more halogen, C₁-C₄alkyl, C₁-C₄haloalkyl, C₁-C₄alkoxy, —CN, —NO₂, or —S(O)₂C₁-C₈alkyl substituents, or
R₄₁ is C₁-C₈alkyl substituted by one or more —COOH, C₁-C₈alkoxycarbonyl, C₁-C₆alkylamino, di(C₁-C₆alkyl)amino or —CN substituents;
R42 is hydrogen, C₁-C₈alkyl, C₃-C₈alkenyl or C₃-C₈alkynyl, or is C₁-C₈alkyl substituted by one or more halogen, —CN or C₁-C₄alkoxy substituents;
R₄₃ and R44 are each independently of the other C₁-C₈alkyl, C₃-C₈alkenyl or C₃-C₈alkynyl, or C₁-C₈alkyl substituted by one or more halogen, —CN or C₁-C₄alkoxy substituents;
R₄₅ is C₁-C₈alkyl, C₁-C₈alkyl substituted by one or more halogen, —CN or C₁-C₄alkoxy substituents, C₃-C₈alkenyl or C₃-C₈alkynyl, or
R₄₅ is phenyl, it being possible for the phenyl ring to be substituted by one or more halogen, C₁-C₄alkyl, C₁-C₄haloalkyl, C₁-C₄alkoxy, —CN, —NO₂, C₁-C₈alkylthio, C₁-C₈alkylsulfinyl or C₁-C₈-alkylsulfonyl substituents;
R₄₆ is hydrogen, C₁-C₈alkyl, C₃-C₈alkenyl, C₃-C₈alkynyl or C₁-C₄haloalkyl;
R₄₇ is hydrogen, C₁-C₈alkyl, C₁-C₄alkoxy, C₃-C₈alkenyl or C₃-C₈alkynyl, or is C₁-C₈alkyl substituted by one or more halogen, —CN, C₁-C₄alkoxy, C₁-C₈alkoxycarbonyl, —NH₂, C₁-C₄alkylamino, di(C₁-C₄-alkyl)amino, —NR₄₈COR₄₉, —NR₅OSO₂R₅₁ or —NR₅₂CO₂R₅₃ substituents, or
R₄₇ is phenyl or benzyl, each of which may in turn be substituted by one or more halogen, C₁-C₄alkyl, C₁-C₄haloalkyl, C₁-C₄alkoxy, —CN, —NO₂, C₁-C₄alkylthio, C₁-C₄alkylsulfinyl or C₁-C₄alkylsulfonyl substituents;
p is 0 or 1;
R₄₈, R49, R₅₀, R₅₁, R₅₂ and R₅₃ are each independently of the others hydrogen, C₁-C₈alkyl, phenyl, benzyl or naphthyl, it being possible for the three last-mentioned aromatic radicals in turn to be substituted by one or more halogen, C₁-C₈alkyl, C₁-C₄haloalkyl, C₁-C₄alkoxy, C₁-C₄alkylamino, di(C₁-C₄alkyl)amino, —NH₂, —CN, —NO₂, C₁-C₄alkylthio, C₁-C₄alkylsulfinyl or C₁-C₄alkylsulfonyl substituents;
R₅₄ and R₅₅ are each independently of the other hydrogen, C₁-C₈alkyl or phenyl, whereby the phenyl ring may in turn be substituted by one or more halogen, C₁-C₄alkyl, C₁-C₄haloalkyl, C₁-C₄alkoxy, —CN, —NO₂, C₁-C₈alkylthio, C₁-C₈alkylsulfinyl or C₁-C₈alkylsulfonyl substituents;
R₅₆ is hydrogen, C₁-C₈alkyl, C₁-C₄haloalkyl, C₁-C₄alkoxy, C₃-C₈alkenyl, C₃-C₈alkynyl or benzyl, it being possible for benzyl in turn to be substituted by one or more halogen, C₁-C₄alkyl, C₁-C₄haloalkyl, C₁-C₄alkoxy, —CN, —NO₂, C₁-C₈alkylthio, C₁-C₈alkylsulfinyl or C₁-C₈alkylsulfonyl substituents;
R₅₇ is C₁-C₈alkyl, C₁-C₄haloalkyl, phenyl, benzyl or naphthyl, it being possible for the three last-mentioned aromatic rings to be substituted by one or more halogen, C₁-C₄alkyl, C₁-C₄haloalkyl, C₁-C₄alkoxy, C₁-C₄alkylamino, di(C₁-C₄alkyl)amino, —NH₂, —CN, —NO₂, C₁-C₄alkylthio, C₁-C₄alkylsulfinyl or C₁-C₄alkylsulfonyl substituents;
R₅₈ and R₅₉ are each independently of the other C₁-C₈alkyl, C₃-C₈alkenyl, C₃-C₈alkynyl, phenyl, benzyl or naphthyl, it being possible for the three last-mentioned aromatic rings to be substituted by one or more halogen, C₁-C₄alkyl, C₁-C₄haloalkyl, C₁-C₄alkoxy, C₁-C₄alkylamino, di(C₁-C₄alkyl)amino, —NH₂, —CN, —NO₂, C₁-C₄alkylthio, C₁-C₄alkylsulfinyl or C₁-C₄alkylsulfonyl substituents;
R₆₀ and R₆, are each independently of the other hydrogen or C₁-C₆alkyl;
R₆₂, R₆₃ and R64 are each independently of the others hydrogen or C₁-C₈alkyl, or
R₆₃ and R₆₄ together form a C₂-C₅alkylene bridge;
R₆₅, R₆₆, R₆₇, R₆₈, R₆₉ and R₇₀ are each independently of the others hydrogen or C₁-C₈alkyl, or R₆₅ and R₆₆ together or R₆₇ and R₆₈ together or R₆₉ and R₇₀ together form a C₂-C₅alkylene bridge; or
each R₂ independently of any others is C₁-C₈alkyl, or is C₁-C₈alkyl mono- or poly-substituted by halogen, —CN, —N₃, —SCN, —NO₂, —NR₇₁R₇₂, —CO₂R₇₃, —CONR₇₄R₇₅, —COR₇₆, —C(R₇₇)═NOR₇₈, —C(S)NR₇₉R₈₀, —C(C₁-C₄alkylthio)═NR₈₁, —OR₈₂, —SR₈₃, —SOR₈₄, —SO₂R₈₅, —O(SO₂)R₈₆, —N(R₈₇)CO₂R₈₈, —N(R₈₉)COR₉₀, —S⁺(R₉₁)₂, —N⁺(R₉₂)₃, —Si(R₉₃)₃ or C₃-C₆cycloalkyl; or
each R₂ independently of any others is C₁-C₈alkyl substituted by a 5- to 7-membered heterocyclic ring system, which may be aromatic or partially or fully saturated and may contain from 1 to 4 hetero atoms selected from nitrogen, oxygen and sulfur, it being possible for that heterocyclic ring system in turn to be substituted by one or more halogen, C₁-C₄alkyl, C₁-C₄haloalkyl, hydroxy-C₁-C₄alkyl, C₁-C₄alkoxy, C₁-C₄alkoxy-C₁-C₄alkyl, —CN, —NO₂, C₁-C₆alkylthio, C₁-C₆alkylsulfinyl or C₁-C₆alkylsulfonyl substituents; or
each R₂ independently of any others is C₂-C₈alkenyl, or is C₂-C₈alkenyl mono- or poly-substituted by halogen, —CN, —NO₂, —CO₂R₉₄, —CONR₉₅R₉₆, —COR₉₇, —C(R₉₈)═NOR₉₉, —C(S)NR₁₀₀R₁₀₁, —C(C₁-C₄alkylthio)═NR₁₀₂, —OR₁₀₃, —Si(R₁₀₄)₃ or C₃-C₆cycloalkyl; or
each R₂ independently of any others is C₂-C₈alkynyl, or is C₂-C₈alkynyl mono- or poly-substituted by halogen, —CN, —CO₂R₁₀₅, —CONR₁₀₆R₁₀₇, —COR₁₀₈, —C(R10 ₉)═NOR₁₁₀, —C(S)NR₁₁₁R₁₁₂, —C(C₁-C₄alkylthio)═NR₁₁₃, —OR₁₁₄, —Si(R₁₁₅)₃ or C₃-C₆cycloalkyl; or
each R₂ independently of any others is C₃-C₆cycloalkyl, or is C₃-C₆cycloalkyl mono- or poly-substituted by halogen, —CN, —CO₂R₁₁₆, —CONR₁₁₇R₁₁₈, —COR₁₁₉, —C(R₁₂₀)═NOR₂₁, —C(S)NR₁₂₂R₁₂₃ or —C(C₁-C₄alkylthio)═NR₁₂₄; or
two adjacent R₂ together form a C₁-C₇alkylene bridge, which may be interrupted by 1 or 2 non-adjacent oxygen atoms and may be substituted by C₁-C₆alkyl or C₁-C₆alkoxy, the total number of ring atoms being at least 5 and at most 9; or
two adjacent R₂ together form a C₂-C₇alkenylene bridge, which may be interrupted by 1 or 2 non-adjacent oxygen atoms and may be substituted by C₁-C₆alkyl or C₁-C₆alkoxy, the total number of ring atoms being at least 5 and at most 9;
R₇₁ is hydrogen or C₁-C₈alkyl;
R₇₂ is hydrogen, C₁-C₈alkyl, C₃-C₈alkenyl, C₃-C₈alkynyl, phenyl or benzyl, wherein phenyl and benzyl may in turn be substituted by one or more halogen, C₁-C₄alkyl, C₁-C₄haloalkyl, C₁-C₄alkoxy, —CN, —NO₂, C₁-C₄alkylthio, C₁-C₄alkylsulfinyl or C₁-C₄alkylsulfonyl substituents; or
R₇₁ and R₇₂ together are a C₂-C₅alkylene chain, which may be interrupted by an oxygen or a sulfur atom;
R₇₃ is hydrogen, C₁-C₈alkyl, C₃-C₈alkenyl or C₃-C₈alkynyl, or is C₁-C₈alkyl, C₃-C₈alkenyl or C₃-C₈alkynyl substituted by one or more halogen, C₁-C₄alkoxy or phenyl substituents, it being possible for phenyl in turn to be substituted by one or more halogen, C₁-C₄alkyl, C₁-C₄haloalkyl, C₁-C₄alkoxy, —CN, —NO₂, C₁-C₄alkylthio, C₁-C₄alkylsulfinyl or C₁-C₄alkylsulfonyl substituents;
R₇₄ is hydrogen or C₁-C₈alkyl;
R₇₅ is hydrogen, C₁-C₈alkyl or C₃-C₇cycloalkyl, or is C₁-C₈alkyl substituted by one or more —COOH, C₁-C₈alkoxycarbonyl, C₁-C₆alkoxy or —CN substituents; or
R₇₅ is C₃-C₈alkenyl, C₃-C₈alkynyl, phenyl or benzyl, wherein phenyl and benzyl may in turn be substituted by one or more halogen, C₁-C₄alkyl, C₁-C₄haloalkyl, C₁-C₄alkoxy, —CN, —NO₂, C₁-C₄alkylthio, C₁-C₄alkylsulfinyl or C₁-C₄alkylsulfonyl substituents; or
R₇₄ and R₇₅ together are a C₂-C₅alkylene chain, which may be interrupted by an oxygen or sulfur atom;
R₇₆ is hydrogen, C₁-C₄alkyl, C₁-C₄haloalkyl or C₃-C₆cycloalkyl;
R₇₇ is hydrogen, C₁-C₄alkyl, C₁-C₄haloalkyl or C₃-C₆cycloalkyl;
R₇₈ is hydrogen, C₁-C₈alkyl, C₃-C₈alkenyl, C₃-C₈alkynyl, C₁-C₄haloalkyl or C₃-C₆haloalkenyl; and
R₇₉ is hydrogen or C₁-C₈alkyl;
R₈₀ is hydrogen or C₁-C₈alkyl, or is C₁-C₈alkyl substituted by one or more —COOH, C₁-C₈alkoxy-carbonyl or —CN substituents; or
R₈₀ is C₃-C₈alkenyl, C₃-C₈alkynyl, phenyl or benzyl, wherein phenyl and benzyl may in turn be substituted by one or more halogen, C₁-C₄alkyl, C₁-C₄haloalkyl, C₁-C₄alkoxy, —CN, —NO₂, C₁-C₄alkylthio, C₁-C₄alkylsulfinyl or C₁-C₄alkylsulfonyl substituents; or
R₇₉ and R₈₀ together are C₂-C₅alkylene;
R₈₁ is hydrogen or C₁-C₈alkyl;
R₈₂ is —Si(C₁-C₆alkyl)₃, C₃-C₈alkenyl, C₃-C₈alkynyl or C₁-C₈alkyl, whereby C₁-C₈alkyl is mono- or poly-substituted by halogen, —CN, —NH₂, C₁-C₆alkylamino, di(C₁-C₆alkyl)amino or C₁-C₄alkoxy;
R₈₃ is hydrogen, C₁-C₈alkyl, C₃-C₈alkenyl, C₃-C₈alkynyl or C₁-C₈alkyl, whereby C₁-C₈alkyl is mono- or poly-substituted by halogen, —CN, —NH₂, C₁-C₆alkylamino, di(C₁-C₆alkyl)amino or C₁-C₄alkoxy;
R₈₄, R₈₅ and R₈₆ are each independently of the others C₁-C₈alkyl, C₃-C₈alkenyl or C₃-C₈alkynyl, or C₁-C₈alkyl which is substituted by one or more halogen, —CN or C₁-C₄alkoxy substituents;
R₈₇ and R₈₉ are each independently of the other hydrogen, C₁-C₈alkyl or C₁-C₈alkoxy;
R₈₈ is C₁-C₈alkyl;
R₉₀ is hydrogen or C₁-C₈alkyl; R₉₁ is C₁-C₄alkyl;
R₉₂ and R₉₃ are each independently of the other C₁-C₆alkyl;
R₉₄ is hydrogen, C₁-C₈alkyl, C₃-C₈alkenyl or C₃-C₈alkynyl, each of which may be mono- or poly-substituted by one or more halogen, C₁-C₄alkoxy or phenyl substituents, wherein phenyl may in turn be substituted by one or more halogen, C₁-C₄alkyl, C₁-C₄haloalkyl, C₁-C₄alkoxy, —CN, —NO₂, C₁-C₄alkylthio, C₁-C₄alkylsulfinyl or C₁-C₄alkylsulfonyl substituents;
R₉₅ is hydrogen or C₁-C₈alkyl;
R₉₆ is hydrogen or C₁-C₈alkyl, or is C₁-C₈alkyl substituted by one or more —COOH, C₁-C₈-alkoxycarbonyl or —CN substituents; or
R₉₆ is C₃-C₈alkenyl, C₃-C₈alkynyl, phenyl or benzyl, wherein phenyl and benzyl may in turn be substituted by one or more halogen, C₁-C₄alkyl, C₁-C₄haloalkyl, C₁-C₄alkoxy, —CN, —NO₂, C₁-C₄alkylthio, C₁-C₄alkylsulfinyl or C₁-C₄alkylsulfonyl substituents; or
R₉₅ and R₉₆ together are C₂-C₅alkylene;
R₉₇ and R₉₈ are each independently of the other hydrogen, C₁-C₄alkyl, C₁-C₄haloalkyl or C₃-C₆cycloalkyl;
R₉₉ is hydrogen, C₁-C₈alkyl, C₃-C₈alkenyl, C₃-C₈alkynyl, C₁-C₄haloalkyl or C₃-C₆haloalkenyl;
R₁₀₀ is hydrogen or C₁-C₈alkyl;
R₁₀₁ is hydrogen or C₁-C₈alkyl, or is C₁-C₈alkyl substituted by one or more —COOH, C₁-C₈-alkoxycarbonyl or —CN substituents; or
R₁₀₁ is C₃-C₈alkenyl, C₃-C₈alkynyl, phenyl or benzyl, wherein phenyl and benzyl may in turn be substituted by one or more halogen, C₁-C₄alkyl, C₁-C₄haloalkyl, C₁-C₄alkoxy, —CN, —NO₂, C₁-C₄alkylthio, C₁-C₄alkylsulfinyl or C₁-C₄alkylsulfonyl substituents; or
R₁₀₀ and R₁₀₁ together are C₂-C₅alkylene; R₁₀₂ is hydrogen or C₁-C₈alkyl;
R₁₀₃ is hydrogen, C₁-C₈alkyl, —Si(C₁-C₆alkyl)₃, C₃-C₈alkenyl or C₃-C₈alkynyl; R₁₀₄ is C₁-C₆alkyl;
R₁₀₅ is hydrogen, C₁-C₈alkyl, C₃-C₈alkenyl or C₃-C₈alkynyl, each of which may be mono- or poly-substituted by one or more halogen, C₁-C₄alkoxy or phenyl substituents, wherein phenyl may in turn be substituted by one or more halogen, C₁-C₄alkyl, C₁-C₄haloalkyl, C₁-C₄alkoxy, —CN, —NO₂, C₁-C₄alkylthio, C₁-C₄alkylsulfinyl or C₁-C₄alkylsulfonyl substituents;
R₁₀₆ is hydrogen or C₁-C₈alkyl; R₁₀₇ is hydrogen or C₁-C₈alkyl, or is C₁-C₈alkyl substituted by one or more —COOH, C₁-C₈alkoxy-carbonyl or —CN substituents; or
R₁₀₇ is C₃-C₈alkenyl, C₃-C₈alkynyl, phenyl or benzyl, wherein phenyl and benzyl may in turn be substituted by one or more halogen, C₁-C₄alkyl, C₁-C₄haloalkyl, C₁-C₄alkoxy, —CN, —NO₂, C₁-C₄alkylthio, C₁-C₄alkylsulfinyl or C₁-C₄alkylsulfonyl substituents; or
R₁₀₆ and R₁₀₇ together are C₂-C₅alkylene;
R₁₀₈ is hydrogen, C₁-C₄alkyl, C₁-C₄haloalkyl or C₃-C₆cycloalkyl;
R₁₀₉ is hydrogen, C₁-C₄alkyl, C₁-C₄haloalkyl or C₃-C₆cycloalkyl;
R₁₁₀ is hydrogen, C₁-C₈alkyl, C₃-C₈alkenyl, C₃-C₈alkynyl, C₁-C₄haloalkyl or C₃-C₆haloalkenyl;
R₁₁₁ is hydrogen or C₁-C₈alkyl;
R₁₁₂ is hydrogen or C₁-C₈alkyl, or is C₁-C₈alkyl substituted by one or more —COOH, C₁-C₈alkoxy-carbonyl or —CN substituents; or
R₁₁₂ is C₃-C₈alkenyl, C₃-C₈alkynyl, phenyl or benzyl, wherein phenyl and benzyl may in turn be substituted by one or more halogen, C₁-C₄alkyl, C₁-C₄haloalkyl, C₁-C₄alkoxy, —CN, —NO₂, C₁-C₄alkylthio, C₁-C₄alkylsulfinyl or C₁-C₄alkylsulfonyl substituents; or
R₁₁₁ and R₁₁₂ together are C₂-C₅alkylene;
R₁₁₃ is hydrogen or C₁-C₈alkyl; R₁₁₄ is hydrogen, C₁-C₈alkyl, —Si(C₁-C₆alkyl)₃, C₃-C₈alkenyl or C₃-C₈alkynyl;
R₁₁₅ is C₁-C₆alkyl; R₁₁₆ is hydrogen, C₁-C₈alkyl, C₃-C₈alkenyl or C₃-C₈alkynyl, each of which may be mono- or poly-substituted by one or more halogen, C₁-C₄alkoxy or phenyl substituents, wherein phenyl may in turn be substituted by one or more halogen, C₁-C₄alkyl, C₁-C₄haloalkyl, C₁-C₄alkoxy, —CN, —NO₂, C₁-C₄alkylthio, C₁-C₄alkylsulfinyl or C₁-C₄alkylsulfonyl substituents;
R₁₁₇ is hydrogen or C₁-C₈alkyl;
R₁₁₈ is hydrogen or C₁-C₈alkyl, or is C₁-C₈alkyl substituted by one or more —COOH, C₁-C₈alkoxy-carbonyl or —CN substituents; or
R₁₁₈ is C₃-C₈alkenyl, C₃-C₈alkynyl, phenyl or benzyl, wherein phenyl and benzyl may in turn be substituted by one or more halogen, C₁-C₄alkyl, C₁-C₄haloalkyl, C₁-C₄alkoxy, —CN, —NO₂, C₁-C₄alkylthio, C₁-C₄alkylsulfinyl or C₁-C₄alkylsulfonyl substituents; or
R₁₁₇ and R₁₁₈ together are C₂-C₅alkylene;
R119 is hydrogen, C₁-C₄alkyl, C₁-C₄haloalkyl or C₃-C₆cycloalkyl;
R₁₂₀ is hydrogen, C₁-C₄alkyl, C₁-C₄haloalkyl or C₃-C₆cycloalkyl;
R₁₂₁ is hydrogen, C₁-C₈alkyl, C₃-C₈alkenyl, C₃-C₈alkynyl, C₁-C₄haloalkyl or C₃-C₆haloalkenyl;
R₁₂₂ is hydrogen or C₁-C₈alkyl; R₁₂₃ is hydrogen or C₁-C₈alkyl, or is C₁-C₈alkyl substituted by one or more —COOH, C₁-C₈alkoxy-carbonyl or —CN substituents; or
R₁₂₃ is C₃-C₈alkenyl, C₃-C₈alkynyl, phenyl or benzyl, wherein phenyl and benzyl may in turn be substituted by one or more halogen, C₁-C₄alkyl, C₁-C₄haloalkyl, C₁-C₄alkoxy, —CN, —NO₂, C₁-C₄alkylthio, C₁-C₄alkylsulfinyl or C₁-C₄alkylsulfonyl substituents; or
R₁₂₂ and R₁₂₃ together are C₂-C₅alkylene; and
R₁₂₄ is hydrogen or C₁-C₈alkyl,
or an agrochemically acceptable salt or any stereoisomer or tautomer of a compound of formula I.

2. A process for the preparation of a compound of formula I according to claim 1, which process comprises reacting a compound of formula II 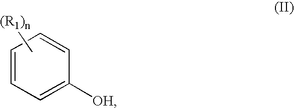
wherein R₁ and n are as defined in claim 1, in the presence of a base, with a compound of formula III 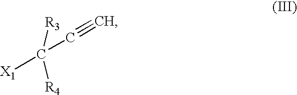
wherein R₃ and R₄ are as defined in claim 1 and X₁ is O-tosyl, O-mesyl, chlorine, bromine or iodine, to form a compound of formula IV 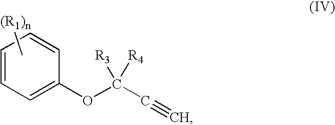
wherein R., R₃, R₄ and n are as defined, and then coupling that compound with a compound of formula V or Va 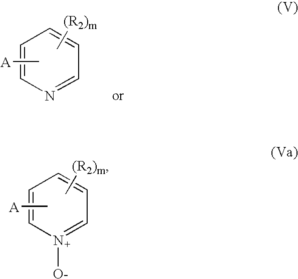
wherein R₂ and m are as defined in claim 1 and A is a leaving group, in the presence of a palladium catalyst, and, if desired, oxidising the resulting pyridine derivative of formula I wherein Z is ═N— to form the corresponding pyridine N-oxide of formula I wherein Z is

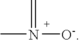

3. A herbicidal and plant-growth-inhibiting composition, comprising a herbicidally effective amount of a compound of formula I according to claim 1 on an inert carrier.

4. A method of controlling undesired plant growth, which method comprises applying a compound of formula I according to claim 1, or a composition comprising such a compound, in a herbicidally effective amount to plants or to the locus thereof.

5. A method of inhibiting plant growth, which method comprises applying a compound of formula I according to cliam 1, or a composition comprising such a compound, in a herbicidally effective amount to plants or to the locus thereof.

6. A compound according to claim 1, wherein Z is ═N—; and each R₂ independently of any others is C₂-C₈alkenyl, or is C₂-C₈alkenyl mono- or poly-substituted by —CN, —NO₂, —CO₂R₉₄, —CONR₉₅R₉₆, —COR₉₇, —C(R₉₈)═NOR₉₉, —C(S)NR₁₀₀R₁₀₁, —C(C₁-C₄alkylthio)═NR₁₀₂, —OR₁₀₃, —Si(R₁₀₄)₃ or C₃-C₆cycloalkyl.

7. A compound according to claim 1, wherein each R₂ independently of any others is halogen, —CN, —SCN, —OCN, —N₃, —CONR₃₆R₃₇, —C(R₃₈)═NOR₃₉, —COR₄₀, —OR₄₁, —SO₂R₄₅, —N([CO]ₚR₄₆)COR₄₇, —N(R₅₆)SO₂R₅₇, —N(SO₂R₅₈)SO₂R₅₉, —N═C(OR₆₀)R₆₁ or C₁-C₈alkyl, or is C₁-C₈alkyl mono- or poly-substituted by halogen, —CN, —N₃, —SCN, —CONR₇₄R₇₅, —COR₇₆, —C(R₇₇)═NOR₇₈, —C(S)NR₇₉R₈₀, —OR₈₂, —SOR₈₄, —SO₂R₈₅ or —N(R₈₉)COR₉₀.

8. A compound according to claim 1, wherein each R₁ independently of any others is halogen, —CN, C₁-C₃alkyl, C₁-C₃haloalkyl, C₁-C₃cyanoalkyl, —OR₁₃ or —C(R₂₄)═NOR₂₅; R₁₃ is C₁-C₃alkyl or di(C₁-C₄-alkyl)amino-C₁-C₄alkyl; R₂₄ is hydrogen or methyl; and R₂₅ is hydrogen or C₁-C₃alkyl.

9. A compound according to claim 1, wherein R₃ and R₄ are each independently of the other hydrogen or methyl.
